SEQUENCE LISTING (55 sequences)

SEQ ID NO: 1 - DNA, 81 bp, synthetic construct
  misc_feature 1..81: note = Description of Artificial Sequence: Synthetic oligonucleotide
  source 1..81: mol_type = other DNA
ccagtgtagactactcaatgctcttacgatcctcacctgctagcacacccatatcccatgcgtactatccacaggtcaacc

SEQ ID NO: 2 - DNA, 81 bp, synthetic construct
  misc_feature 1..81: note = Description of Artificial Sequence: Synthetic oligonucleotide
  source 1..81: mol_type = other DNA
ccagtgtagactactcaatgcgggttgcgacatggtggtaagagctcagcccgttcccatagtactatccacaggtcaacc

SEQ ID NO: 3 - DNA, 81 bp, synthetic construct
  misc_feature 1..81: note = Description of Artificial Sequence: Synthetic oligonucleotide
  source 1..81: mol_type = other DNA
ccagtgtagactactcaatgcacggcctgttcgtaagacccttacagactagtttttccctgtactatccacaggtcaacc

SEQ ID NO: 4 - DNA, 81 bp, synthetic construct
  misc_feature 1..81: note = Description of Artificial Sequence: Synthetic oligonucleotide
  source 1..81: mol_type = other DNA
ccagtgtagactactcaatgccctattagctgtatcgatccgtttagtcgctcctccgatagtactatccacaggtcaacc

SEQ ID NO: 5 - DNA, 81 bp, synthetic construct
  misc_feature 1..81: note = Description of Artificial Sequence: Synthetic oligonucleotide
  source 1..81: mol_type = other DNA
ccagtgtagactactcaatgcctggtaaatcgatgaccgctgcctcgcctgagtaatcatcgtactatccacaggtcaacc

SEQ ID NO: 6 - DNA, 81 bp, synthetic construct
  misc_feature 1..81: note = Description of Artificial Sequence: Synthetic oligonucleotide
  source 1..81: mol_type = other DNA
ccagtgtagactactcaatgccgtggactggtcgggtttggattcggcagatgaatcagtagtactatccacaggtcaacc

SEQ ID NO: 7 - DNA, 81 bp, synthetic construct
  misc_feature 1..81: note = Description of Artificial Sequence: Synthetic oligonucleotide
  source 1..81: mol_type = other DNA
ccagtgtagactactcaatgccttgtaagaagaacaatcgccgcttcgcctgaataggttcgtactatccacaggtcaacc

SEQ ID NO: 8 - DNA, 81 bp, synthetic construct
  misc_feature 1..81: note = Description of Artificial Sequence: Synthetic oligonucleotide
  source 1..81: mol_type = other DNA
ccagtgtagactactcaatgcggaccgttgcctcgcccgagtaatccgccatcgcctttccgtactatccacaggtcaacc

SEQ ID NO: 9 - DNA, 81 bp, synthetic construct
  misc_feature 1..81: note = Description of Artificial Sequence: Synthetic oligonucleotide
  source 1..81: mol_type = other DNA
ccagtgtagactactcaatgcttaagttctggggacacgtgatgaacgcatttaatggggcgtactatccacaggtcaacc

SEQ ID NO: 10 - DNA, 81 bp, synthetic construct
  misc_feature 1..81: note = Description of Artificial Sequence: Synthetic oligonucleotide
  source 1..81: mol_type = other DNA
ccagtgtagactactcaatgccgtggactggtcgggtttggattcggcagatgaatcactagtactatccacaggtcaacc

SEQ ID NO: 11 - DNA, 81 bp, synthetic construct
  misc_feature 1..81: note = Description of Artificial Sequence: Synthetic oligonucleotide
  source 1..81: mol_type = other DNA
ccagtgtagactactcaatgcggctgtgtgacttgacctttggaatgggtgggagggatgggtactatccacaggtcaacc

SEQ ID NO: 12 - DNA, 76 bp, synthetic construct
  misc_feature 1..76: note = Description of Artificial Sequence: Synthetic oligonucleotide
  source 1..76: mol_type = other DNA
ccagtgtagactactcaatgcggtgtggtgaccttgacctatggaacctggttgtagtactatccacaggtcaacc

SEQ ID NO: 13 - DNA, 82 bp, synthetic construct
  misc_feature 1..82: note = Description of Artificial Sequence: Synthetic oligonucleotide
  source 1..82: mol_type = other DNA
ccagtgtagactactcaatgctcgacatttccgccccgacggccctcctagtgatggggagagtactatccacaggtcaacc

SEQ ID NO: 14 - DNA, 81 bp, synthetic construct
  misc_feature 1..81: note = Description of Artificial Sequence: Synthetic oligonucleotide
  source 1..81: mol_type = other DNA
ccagtgtagactactcaatgccttccattcacctaccgagctaagcgttcgacttaggtctgtactatccacaggtcaacc

SEQ ID NO: 15 - protein, 305 aa, Clostridium difficile
MENNKCREDFRFTQEYEEDYPNTNERYYENYQVADRYYNYPNKYKEPKIKQCCCKKSMREALELLRYDALRPFVNFNQFAFISDFFIVGANLVGIDLSAPPKDNLSGLDGTFERFSACNCDLIDIAGRVSYPIPVPLTLEGLINTIGTIPGVAELIALIDAVIPPTIDLGAILDAILAAIIDFILAASTPLANVDLASLCNLKAVAFDITPADYEDFIASLGYYLDKKHYKECNCNCDCDDCCCNKGILDNLYMSNINNQVTVVAGSLVLTGVEVLGKKNDVIVLGNSNDSRIYFVCVDSIDYIA

SEQ ID NO: 16 - protein, 712 aa, Clostridium difficile
MIYMPNLPSLGSKAPDFKANTTNGPIRLSDYKGNWIVLFSHPGDFTPVCTTEFLCFAKYYDEFKKRNTELIGLSVDSNSSHLAWMYNISLLTGVEIPFPIIEDRDMRIAKLYGMISKPMSDTSTVRSVFIIDNNQILRTILYYPLTTGRNIPEILRIVDALQTSDRDNIVTPANWFPGMPVILPYPKNYKELKNRVNSCNKKYSCMDWYLCFVPDNYNDEEVSKKIDNTCSWKKEHTKNIENECNCEHEHHDYLNKALDCKQEHKTDIKDDCNHEKKHTKNTNKVHNSKQDKFKDKSCDEMNFNYDKDESCDKINSSYNKEDSSYEDFYKHNYKNYDYTSEKNTKKIAMKTLKDSKKLVRPQITDPYNPIVENANCPDINPIVAEYVLGNPTNVDAQLLDAVIFAFAEIDQSGNLFIPYPRFLNQLLALKGEKPSLKVIVAIGGWGAEGFSDAALTPTSRYNFARQVNQMINEYALDGIDIDWEYPGSSASGITSRPQDRENFTLLLTAIRDVIGDDKWLSVAGTGDRGYINSSAEIDKIAPIIDYFNLMSYDFTAGETGPNGRKHQANLFDSDLSLPGYSVDAMVRNLENAGMPSEKILLGIPFYGRLGATITRTYDELRRDYINKNGYEYRFDNTAQVPYLVKDGDFAMSYDDALSIFLKTQYVLRNCLGGVFSWTSTYDQANILARTMSIGINDPEVLKEELEGIYGQF

SEQ ID NO: 17 - protein, 332 aa, Clostridium difficile
PIVAEYVLGNPTNVDAQLLDAVIFAFAEIDQSGNLFIPYPRFLNQLLALKGEKPSLKVIVAIGGWGAEGFSDAALTPTSRYNFARQVNQMINEYALDGIDIDWEYPGSSASGITSRPQDRENFTLLLTAIRDVIGDDKWLSVAGTGDRGYINSSAEIDKIAPIIDYFNLMSYDFTAGETGPNGRKHQANLFDSDLSLPGYSVDAMVRNLENAGMPSEKILLGIPFYGRLGATITRTYDELRRDYINKNGYEYRFDNTAQVPYLVKDGDFAMSYDDALSIFLKTQYVLRNCLGGVFSWTSTYDQANILARTMSIGINDPEVLKEELEGIYGQF

SEQ ID NO: 18 - protein, 405 aa, Clostridium difficile
MQDYKKNKRRMMNQPMSTMNEEEVYTDEINSEDMRGFKKSHHHNGCNTDNKCECHDDCNPCNPCNPCKPNPCNPCKPNPCDDNCGCHDNCKCDCEPCEMDSDECFENKCGPECCNPISPRNFSVSNAVPFAIEANRIFDTMQFQTFTDATGPNGEPLTFETEVVEVFGSVPSAGQASVTIEKICLSNDGIVIDTGMTTLEDFDLDPLGDIVGRNCETTFEFAVCGERNSECCRQGKGKSVAYKQRGLTVAVRNLVLELRGRCGCTEFVALAFPAVRAGGGCKRRVDYVEFTFNTLSAPICLPADGRAVTLRQEYQTNLTVDCIGKSILKLECNECCEPFYELIIPNDIDLVLCLQETVSTLISEQIVVLASPNPIQPRLVDTFSKVCDFSQCGPNHGSGKPSCHR

SEQ ID NO: 19 - protein, 157 aa, Clostridium difficile
MENKKCYSEDWYERGESTAKWFQNDREEYEREAYDEDRERRGSNCGCSDSGENRPRNCERFRREAEIREREAREAFCESSEKKKEALAYECEARKLWEEAEKYWDEYSKYNYKGIEYLAEAARLFDEGMECEARRNGNNGGNNNNCCHKCNCNCCRK

SEQ ID NO: 20 - protein, 432 aa, Clostridium difficile
NTNKVHNSKQDKFKDKSCDEMNFNYDKDESCDKINSSYNKEDSSYEDFYKHNYKNYDYTSEKNTKKIAMKTLKDSKKLVRPQITDPYNPIVENANCPDINPIVAEYVLGNPTNVDAQLLDAVIFAFAEIDQSGNLFIPYPRFLNQLLALKGEKPSLKVIVAIGGWGAEGFSDAALTPTSRYNFARQVNQMINEYALDGIDIDWEYPGSSASGITSRPQDRENFTLLLTAIRDVIGDDKWLSVAGTGDRGYINSSAEIDKIAPIIDYFNLMSYDFTAGETGPNGRKHQANLFDSDLSLPGYSVDAMVRNLENAGMPSEKILLGIPFYGRLGATITRTYDELRRDYINKNGYEYRFDNTAQVPYLVKDGDFAMSYDDALSIFLKTQYVLRNCLGGVFSWTSTYDQANILARTMSIGINDPEVLKEELEGIYGQF

SEQ ID NO: 21 - DNA, 21 bp, synthetic construct
  misc_feature 1..21: note = Description of Artificial Sequence: Synthetic oligonucleotide
  source 1..21: mol_type = other DNA
ccagtgtagactactcaatgc

SEQ ID NO: 22 - DNA, 20 bp, synthetic construct
  misc_feature 1..20: note = Description of Artificial Sequence: Synthetic oligonucleotide
  source 1..20: mol_type = other DNA
gtactatccacaggtcaacc

SEQ ID NO: 23 - DNA, 36 bp, synthetic construct
  misc_feature 1..36: note = Description of Artificial Sequence: Synthetic oligonucleotide
  source 1..36: mol_type = other DNA
atcgatgaccgctgcctcgcctgagtaatcatcgta

SEQ ID NO: 24 - DNA, 33 bp, synthetic construct
  misc_feature 1..33: note = Description of Artificial Sequence: Synthetic oligonucleotide
  source 1..33: mol_type = other DNA
ccatactcaatgctcttacgatcctcatcaacc

SEQ ID NO: 25 - DNA, 41 bp, synthetic construct
  misc_feature 1..41: note = Description of Artificial Sequence: Synthetic oligonucleotide
  source 1..41: mol_type = other DNA
ccagtgtagactactcaatgctcttacgatcctcatcaacc

SEQ ID NO: 26 - DNA, 38 bp, synthetic construct
  misc_feature 1..38: note = Description of Artificial Sequence: Synthetic oligonucleotide
  source 1..38: mol_type = other DNA
agtgtagactactcaatgcggctggccacaggtcaacc

SEQ ID NO: 27 - DNA, 50 bp, synthetic construct
  misc_feature 1..50: note = Description of Artificial Sequence: Synthetic oligonucleotide
  source 1..50: mol_type = other DNA
cttgacctttggaatgggtgggagggatgggtactatccacaggtcaacc

SEQ ID NO: 28 - DNA, 24 bp, synthetic construct
  misc_feature 1..24: note = Description of Artificial Sequence: Synthetic oligonucleotide
  source 1..24: mol_type = other DNA
aatgggtgggagggatgggtacta

SEQ ID NO: 29 - DNA, 51 bp, synthetic construct
  misc_feature 1..51: note = Description of Artificial Sequence: Synthetic oligonucleotide
  source 1..51: mol_type = other DNA
cttgacctttggaatgggtagggagggagggatactatccacaggtcaacc

SEQ ID NO: 30 - DNA, 46 bp, synthetic construct
  misc_feature 1..46: note = Description of Artificial Sequence: Synthetic oligonucleotide
  source 1..46: mol_type = other DNA
cttgacctttggaatgggtgggagggagggtatccacaggtcaacc

SEQ ID NO: 31 - DNA, 57 bp, synthetic construct
  misc_feature 1..57: note = Description of Artificial Sequence: Synthetic oligonucleotide
  source 1..57: mol_type = other DNA
actactcaatgctcgacatttccgccccgacggccctcctagtgaggggagagtaga

SEQ ID NO: 32 - DNA, 58 bp, synthetic construct
  misc_feature 1..58: note = Description of Artificial Sequence: Synthetic oligonucleotide
  source 1..58: mol_type = other DNA
actactcaatgctcgacatttccgccccgacggccctcctagtgatggggagagtaga

SEQ ID NO: 33 - DNA, 48 bp, synthetic construct
  misc_feature 1..48: note = Description of Artificial Sequence: Synthetic oligonucleotide
  source 1..48: mol_type = other DNA
actcaaggccgtggactggtcgggtttggattcggcagatgaatcact

SEQ ID NO: 34 - DNA, 31 bp, synthetic construct
  misc_feature 1..31: note = Description of Artificial Sequence: Synthetic oligonucleotide
  source 1..31: mol_type = other DNA
actcaaggccgtggactggtcgggtttggat

SEQ ID NO: 35 - DNA, 25 bp, synthetic construct
  misc_feature 1..25: note = Description of Artificial Sequence: Synthetic oligonucleotide
  source 1..25: mol_type = other DNA
acccgtgggactgggtcgggtcggg

SEQ ID NO: 36 - DNA, 62 bp, synthetic construct
  misc_feature 1..62: note = Description of Artificial Sequence: Synthetic oligonucleotide
  source 1..62: mol_type = other DNA
aactgcctggtaaatcgatgaccgctgcctcgcctgagtaatcatcgtactatccacaggtc

SEQ ID NO: 37 - DNA, 41 bp, synthetic construct
  misc_feature 1..41: note = Description of Artificial Sequence: Synthetic oligonucleotide
  source 1..41: mol_type = other DNA
gtaaatcgatgaccgctgcctcgcctgagtaatcatcgtac

SEQ ID NO: 38 - DNA, 56 bp, synthetic construct
  misc_feature 1..56: note = Description of Artificial Sequence: Synthetic oligonucleotide
  source 1..56: mol_type = other DNA
actactcaaacccgtggactggtcgggtttggattcggcagatgaatcagtagaaa

SEQ ID NO: 39 - DNA, 57 bp, synthetic construct
  misc_feature 1..57: note = Description of Artificial Sequence: Synthetic oligonucleotide
  source 1..57: mol_type = other DNA
actactcaatgccgtggactggtcgggtttggaatcggcagatgaatcagtagtaaa

SEQ ID NO: 40 - DNA, 16 bp, synthetic construct
  misc_feature 1..16: note = Description of Artificial Sequence: Synthetic oligonucleotide
  source 1..16: mol_type = other DNA
cattccaaaggtcaag

SEQ ID NO: 41 - DNA, 21 bp, synthetic construct
  misc_feature 1..21: note = Description of Artificial Sequence: Synthetic oligonucleotide
  source 1..21: mol_type = other DNA
cacacattccaaaaggtcaag

SEQ ID NO: 42 - DNA, 53 bp, synthetic construct
  misc_feature 1..53: note = Description of Artificial Sequence: Synthetic oligonucleotide
  source 1..53: mol_type = other DNA
ctcaatgccttccattcacctaccgagctaagcgttcgacttaggtctgtact

SEQ ID NO: 43 - DNA, 35 bp, synthetic construct
  misc_feature 1..35: note = Description of Artificial Sequence: Synthetic oligonucleotide
  source 1..35: mol_type = other DNA
cctaccgagctaagcgttcgacttaggtctgtact

SEQ ID NO: 44 - DNA, 48 bp, synthetic construct
  misc_feature 1..48: note = Description of Artificial Sequence: Synthetic oligonucleotide
  source 1..48: mol_type = other DNA
ctcacctgctagcacacccatatcccatgggtacaatccacaggtcaa

SEQ ID NO: 45 - DNA, 48 bp, synthetic construct
  misc_feature 1..48: note = Description of Artificial Sequence: Synthetic oligonucleotide
  source 1..48: mol_type = other DNA
ctcacctgctagcacacccacatcccgtgcgtgctatccacaggtgaa

SEQ ID NO: 46 - DNA, 77 bp, synthetic construct
  misc_feature 1..77: note = Description of Artificial Sequence: Synthetic oligonucleotide
  source 1..77: mol_type = other DNA
agtgcagactactcaatgcacggccggttcggaagacccttccagactagtttttccctgtactagtccaccggcta

SEQ ID NO: 47 - DNA, 50 bp, synthetic construct
  misc_feature 1..50: note = Description of Artificial Sequence: Synthetic oligonucleotide
  source 1..50: mol_type = other DNA
agtgcagactactcaatgcacggcctggttcgtaagacccttaccagact

SEQ ID NO: 48 - DNA, 61 bp, synthetic construct
  misc_feature 1..61: note = Description of Artificial Sequence: Synthetic oligonucleotide
  source 1..61: mol_type = other DNA
cggttgcgacatggtggtaagagctcagcccgttcccatagtactatccacaggtcaacct

SEQ ID NO: 49 - DNA, 63 bp, synthetic construct
  misc_feature 1..63: note = Description of Artificial Sequence: Synthetic oligonucleotide
  source 1..63: mol_type = other DNA
cggttgcgacatggtggtaagagctcagcccgttcccatagtactatccacaggtcgcaacct

SEQ ID NO: 50 - DNA, 76 bp, synthetic construct
  misc_feature 1..76: note = Description of Artificial Sequence: Synthetic oligonucleotide
  source 1..76: mol_type = other DNA
cccgtgtagactactcaatgcgggctgcgacatggtggtaagagctcagcccgttcccatagtactatccacgggt

SEQ ID NO: 51 - DNA, 32 bp, synthetic construct
  misc_feature 1..32: note = Description of Artificial Sequence: Synthetic oligonucleotide
  source 1..32: mol_type = other DNA
cccgtgtagactattttagtactatccacggg

SEQ ID NO: 52 - DNA, 37 bp, synthetic construct
  misc_feature 1..37: note = Description of Artificial Sequence: Synthetic oligonucleotide
  source 1..37: mol_type = other DNA
tgcgggctgcgacatggtggtaagagctcagcccgtt

SEQ ID NO: 53 - DNA, 84 bp, synthetic construct
  misc_feature 1..84: note = Description of Artificial Sequence: Synthetic oligonucleotide
  source 1..84: mol_type = other DNA
acccaggtgtaggacgactcaatgccctattagctgtatcgatccgtttagtcgctcctccgatagtaccctatccaccaggga

SEQ ID NO: 54 - DNA, 81 bp, synthetic construct
  misc_feature 1..81: note = Description of Artificial Sequence: Synthetic oligonucleotide
  source 1..81: mol_type = other DNA
  modified_base 72: mod_base = OTHER; note = Uracil
accaggtggtagacctactcacatgccctattagcgtgtatcgatccggtttagtccgcttcgatagtagtcccaccagga

SEQ ID NO: 55 - DNA, 53 bp, synthetic construct; identical to SEQ ID NO: 42
  misc_feature 1..53: note = Description of Artificial Sequence: Synthetic oligonucleotide
  source 1..53: mol_type = other DNA

(19) United States
(12) Patent Application Publication
(10) Pub. No.: US 2026/0354387 A1
(43) Pub. Date: Nov. 12, 2026

(54) COMPOSITIONS AND METHODS OF REDUCING OR PREVENTING CLOSTRIDIOIDES DIFFICILE COLONIZATION

(71) Applicant: LIV PROCESS, INC., Ardmore, PA (US)

(72) Inventors: Ronald J. Shannon, Ardmore, PA (US); Michael McIntyre, Ardmore, PA (US); Stefania Fabbri, Daresbury (GB); Samantha Westgate, Daresbury (GB)

(73) Assignee: LIV PROCESS, INC., Ardmore, PA (US)

(21) Appl. No.: 19/742,169

(22) Filed: Jul. 15, 2026

Related U.S. Application Data
(62) Division of application No. 17/996,394, filed on Apr. 16, 2021, now Pat. No. 12,708,119.
(60) Provisional application No. 63/011,850, filed on Apr. 17, 2020.

(51) Int. Cl. A01N 63/60; A01N 37/16; C12N 15/115

(52) U.S. Cl. CPC A01N 63/60; A01N 37/16; C12N 15/115; C12N 2310/16

(57) ABSTRACT

Embodiments of the present disclosure relate to a method of preventing and/or reducing C. difficile colonization of a surface. In certain embodiments, the aptamer can be used to kill and/or deactivate a C. difficile spore. In certain embodiments, the aptamer can be used to increase the sporicidal activity of a sporicidal agent. Embodiments of the present disclosure relate to the use of aptamers which specifically bind to C. difficile surface proteins, for example proteins located on the surface of a C. difficile spore.

### RELATED APPLICATIONS

This application is a divisional application of U.S. application Ser. No. 17/996,394, filed Oct. 17, 2022, which is a U.S. 371 National Stage Entry of PCT International Application No. PCT/US2021/027601, filed Apr. 16, 2021, which claims the benefit of and priority to U.S. Provisional Application No. 63/011,850, filed Apr. 17, 2020, the entire disclosure of which is incorporated herein by reference in its entirety.

### SEQUENCE LISTING

The instant application contains a Sequence Listing which has been submitted electronically in XML file format and is hereby incorporated by reference in its entirety. Said XML copy, created on Jul. 14, 2026, is named 193519-010133_DIV_SL.xml and is 74,641 bytes in size.

### FIELD

Embodiments of the present disclosure relate to a method of preventing and/or reducing C. difficile colonization of a surface. Embodiments of the present disclosure relate to the use of aptamers which specifically bind to C. difficile surface proteins e.g. proteins located on the surface of a C. difficile spore. In certain embodiments, the aptamer may be used to kill and/or deactivate a C. difficile spore. In certain embodiments, the aptamer may be used to increase the sporicidal activity of a sporicidal agent e.g. a sporicidal solution.

### BACKGROUND

Clostridioides difficile (also referred to as C. difficile and previously referred to as Clostridium difficile) is a Gram-positive, anaerobic spore former and is an important nosocomial and community-acquired pathogenic bacterium. C. difficile infections (CDI) are a leading cause of infections worldwide with elevated rates of morbidity. Despite the fact that two major virulence factors, the enterotoxin TcdA and the cytotoxin TcdB, are essential in the development of CDI, C. difficile spores are the main vehicle of infection, and persistence and transmission of CDI and are thought to play an essential role in episodes of CDI recurrence and horizontal transmission.

### SUMMARY

Aspects of the present disclosure relates to a method for reducing and/or preventing Clostridioides difficile (C. difficile) contamination and products and kits for use in the method. In some embodiments, a method of killing and/or inactivating C. difficile spores comprising contacting the spores with an aptamer capable of specifically binding to C. difficile, and an agent selected from the group consisting of a sporicidal agent and a sporostatic agent, is provided. In some embodiments, a method of enhancing the sporicidal activity against C. difficile spores of a sporicidal agent comprising contacting the spores with an aptamer capable of specifically binding to C. difficile, and an agent selected from the group consisting of a sporicidal agent and a sporostatic agent, is provided. In some embodiments, the agent is a sporicidal agent.

In some embodiments, kits for killing and/or inactivating C. difficile spores are provided. In some embodiments, the kit comprises an aptamer capable of specifically binding to C. difficile, and an agent selected from the group consisting of a sporicidal agent and a sporostatic agent. In some embodiments, kits for enhancing the sporicidal activity against C. difficile spores are provided. In some embodiments, the kit comprises an aptamer capable of specifically binding to C. difficile, and a sporicidal agent and a sporostatic agent. In some embodiments, the kit comprises a light source and viewing goggles for determining the presence, absence and/or concentration of C. difficile at the location prior to contacting the location with the sporicidal agent and/or sporostatic agent.

Embodiments of the disclosure relate to a method of killing and/or inactivating C. difficile spores located on a surface. The surface may be a surface located in a hospital or other healthcare facility such as a care home. The surface may be for example a sheet, a hospital surgical gown, flooring, walls, an operating table, furniture or the like. Embodiments of the present disclosure relate to methods of enhancing the sporicidal activity of one or more sporicidal agents comprising the use of aptamers described herein. Embodiments of the disclosure relate to the reduction or prevention of colonization of a location e.g. a surface by C. difficile.

In some embodiments, 99.9% of the spores are not capable of transforming into vegetative cells. In an embodiment, 99.99% or 99.999% of the spores are not capable of transforming into vegetative cells. In some embodiments, at least 85% of the spores are not capable of transforming into vegetative cells.

In certain embodiments, the disclosure relates to the use of one or more aptamers, e.g. one, two, three or more aptamers capable of binding to a C. difficile spore to facilitate the kill and/or deactivate a C. difficile spore. The aptamer(s) may be for use in isolation and/or in combination with a sporicidal agent. Embodiments of the disclosure may comprise the use of one or more aptamers as described herein to increase the sporicidal activity of a sporicidal agent. Further details of sporicidal fluids for use according to embodiments of the disclosure are provided herein.

The C. difficile colonization may be comprised of a single strain or may be a mixture of strains.

In an aspect of the present disclosure there is provided a method for killing or inactivating C. difficile spores, comprising contacting the spores with an agent selected from the group consisting of a sporicidal agent and a sporostatic agent and one or more aptamers capable of specifically binding to C. difficile.

In an aspect of the present disclosure there is provided a method for (i) detecting the presence of C. difficile at a location and (ii) killing or inactivating C. difficile spores, comprising contacting the spores with an agent selected from the group consisting of a sporicidal agent and a sporostatic agent and one or more aptamers capable of specifically binding to C. difficile. In embodiments, one or more aptamers comprises a detection molecule as described herein.

In an aspect of the present disclosure there is provided a method for killing or inactivating C. difficile spores, comprising contacting the spores with an agent selected from the group consisting of a sporicidal agent and a sporostatic agent and one or more aptamers capable of specifically binding to C. difficile. In embodiments, one or more aptamers comprises a detection molecule as described herein.

In an aspect of the present disclosure there is provided a method for enhancing a sporicidal effect against C. difficile of a sporicidal agent, the method comprising contacting C. difficile spores with one or more aptamer capable of specifically binding to C. difficile and an agent selected from the group consisting of a sporicidal agent and a sporostatic agent.

In an aspect of the present disclosure there is provided a method for (i) detecting the presence of C. difficile at a location and (ii) enhancing a sporicidal and/or sporostatic effect against C. difficile of a sporicidal agent or sporostatic agent, the method comprising contacting C. difficile spores with one or more aptamers capable of specifically binding to C. difficile and an agent selected from the group consisting of a sporicidal agent and a sporostatic agent. In embodiments, the one or more aptamers comprises a detection molecule as described herein.

In embodiments, the agent is a sporicidal agent.

In embodiments, the method comprises locating the one or more aptamers and the sporicidal agent at a location suspected of comprising C. difficile spores.

In embodiments, the location comprises a surface and the method comprises contacting the surface with the sporicidal agent prior to, at essentially the same time, or subsequent to the one or more aptamers.

In embodiments, the method comprises the steps of contacting the surface with the one or more aptamers for a predetermined period of time configured to enable the one or more aptamers to bind to the C. difficile spores to form one or more aptamer-spore complexes, and contacting the surface comprising the one or more aptamer-spore complexes with the sporicidal agent.

In embodiments, the method comprises determining the one or more aptamers bound to the C. difficile spore prior to contacting the surface with the sporicidal agent.

In embodiments, the method further comprises a step of contacting the surface with a GH neutraliser.

In embodiments, the surface is a surface located on an object e.g. a hospital bed, an operating table, clothing, general-premise surfaces (e.g. floors, walls, ceilings, exterior of furniture), specific-equipment surfaces (e.g. hard surfaces, manufacturing equipment, processing equipment, etc.), textiles (e.g. cottons, wools, silks, synthetic fabrics such as polyesters, polyolefins, and acrylics, fiber blends such as cotton polyester, etc.), wood and cellulose-based systems (e.g. paper), soil, animal carcasses (e.g. hide, meat, hair, feathers, etc.). In some embodiments, the object can be foodstuffs (e.g. fruits, vegetables, nuts, meats, etc.).

In embodiments, the method comprises the steps of contacting a media with the one or more aptamers for a predetermined period of time configured to enable the one or more aptamers to bind to the C. difficile spores to form an aptamer-spore complex, and contacting the media comprising the aptamer-spore complex with the sporicidal agent. In some embodiments the media is water.

In embodiments, the method comprises submerging the spores with a composition comprising the one or more aptamers and/or submerging the spores in the agent.

In embodiments, the method comprises spraying the C. difficile spores with a composition comprising the aptamer and/or spraying the C. difficile spores with the agent.

In embodiments, the method comprises applying a composition comprising the aptamer by means of a cloth and/or applying the agent by means of a cloth and/or a wipe.

In embodiments, the method is performed at a temperature of between about 5 °C to about 90 °C e.g. 5 °C, 10 °C, 15 °C, 20 °C, 25 °C, 30 °C, 35 °C, 40 °C, 45 °C, 50 °C, 55 °C, 60 °C, 65 °C, 70 °C, 75 °C, 80 °C or 90 °C. In embodiments, the method may be performed at a temperature in the range of about 5 °C to about 90 °C. In embodiments, the method may be performed at a temperature in the range of about 5 °C to 10 °C, 10 °C to 15 °C, 15 °C to 20 °C, 20 °C to 25 °C, 25 °C to 30 °C, 30 °C to 35 °C, 35 °C to 40 °C, 40 to 45 °C, 45 °C to 50 °C, 50 °C to 55 °C, 55 °C to 60 °C, 60 °C to 65 °C, 65 °C to 70 °C, 70 °C to 75 °C, 75 °C to 80 °C, 80 °C to 85 °C, 85 °C to 90 °C, or any interval between any of these temperature ranges. In an embodiment, the heating temperatures are selected from 1 °C increments selected from the range of 5 °C to 90 °C.

In embodiments, the C. difficile spores comprise a protein, wherein the protein is a spore coat surface protein or an exosporium layer protein.

In embodiments, the C. difficile protein selected from CdeC, CdeM, CotA, CotE and CotE Chitinase.

In embodiments, the C. difficile protein is a CdeC protein having an amino acid sequence as set forth in SEQ. ID. NO 18.

In embodiments, the C. difficile protein is a CdeM protein having an amino acid sequence as set forth in SEQ. ID. NO. 19.

In embodiments, the C. difficile protein is a CotA, protein having an amino acid sequence as set forth in SEQ. ID. NO. 15.

In embodiments, the C. difficile protein is a CotE, protein having an amino acid sequence as set forth in SEQ. ID. NO. 16.

In embodiments, the C. difficile protein is a CotE Chitinase protein having an amino acid sequence as set forth in SEQ. ID. NO. 17.

In embodiments, the C. difficile protein is rCotE protein having an amino acid sequence as set forth in SEQ. ID. NO. 20.

In embodiments, the aptamer is a single stranded DNA aptamer.

In embodiments, the aptamer comprises, consists essentially of, or consists of:
(a) a nucleic acid sequence selected from any one of the nucleic acid sequences as set forth in any of SEQ ID NOs: 1 to 14, 23 to 39 or 43-55;
(b) a nucleic acid sequence having at least 85% identity for example 90%, 95%, 96%, 97%, 98% or 99% sequence identity with any one of the nucleic acid sequences as set forth in any of SEQ ID NOs: 1 to 14, 23 to 39 or 43-55;
(c) a nucleic acid sequence having at least about 20 consecutive nucleotides of any one the nucleic acid sequences as set forth in any of SEQ ID NOs: 1 to 14, 23 to 39 or 43-55; or
(d) a nucleic acid sequence having at least about 20 consecutive nucleotides of a sequence having at least 85% identity with any one of SEQ ID NOs 1 to 14, 23 to 39 or 43-55.

In embodiments, the method comprises contacting the location with a plurality of aptamers, wherein each aptamer is capable of specifically binding to a C. difficile spore. In embodiments, the plurality of aptamers comprises at least two aptamers capable of specifically binding to the same epitope of a C. difficile spore. In embodiments, the plurality of aptamers comprises at least two aptamers, each aptamer being capable of specifically binding to a different epitope of a C. difficile spore.

In embodiments, the plurality of aptamers comprises at least two aptamers selected from the group consisting of an aptamer comprising, consisting essentially of, or consisting of:
(a) a nucleic acid sequence selected from any one of the nucleic acid sequences as set forth in any of SEQ ID NOs: 1 to 14, 23 to 39 or 43-55;
(b) a nucleic acid sequence having at least 85% identity for example 90%, 95%, 96%, 97%, 98% or 99% sequence identity with any one of the nucleic acid sequences as set forth in any of SEQ ID NOs: 1 to 14, 23 to 39 or 43-55;
(c) a nucleic acid sequence having at least about 20 consecutive nucleotides of any one the nucleic acid sequences as set forth in any of SEQ ID NOs: 1 to 14, 23 to 39 or 43-55;
(d) a nucleic acid sequence having at least about 20 consecutive nucleotides of a sequence having at least 85% identity with any one of SEQ ID NOs: 1 to 14, 23 to 39 or 43-55, or
(e) a combination of any of (a) to (d).

In embodiments, the plurality of aptamers comprises at least two aptamers selected from the group consisting of:
a) an aptamer which specifically binds to a CdeC;
b) an aptamer which specifically binds to CdeM,
c) an aptamer which specifically binds to CotA,
d) an aptamer which specifically binds to CotE; and
e) an aptamer which specifically binds to CotE Chitinase;
f) a combination of any of (a) to (e).

In embodiments, the plurality of aptamers comprises an aptamer comprising, consisting essentially of, or consisting of a nucleic acid sequence as set forth in SEQ. ID. NO. 1; an aptamer comprising, consisting essentially of, or consisting of a nucleic acid sequence as set forth in SEQ. ID. NO. 5; and an aptamer comprising, consisting essentially of, or consisting of a nucleic acid sequence as set forth in SEQ. ID. NO. 6.

In embodiments, the aptamer comprises a detectable molecule. In embodiments, the detectable molecule is a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, or a liposome.

In certain embodiments, the aptamer comprises one or both molecules of a FRET pair.

In embodiments, the agent is a sporicidal solution which comprises chlorhexidine gluconate and optionally isopropyl alcohol.

In embodiments, the agent is a sporicidal agent which comprises peracetic acid.

In embodiments, the agent is a peracetic acid generating agent, and wherein the method further comprises wetting a wipe comprising sodium percarbonate with an aqueous solution prior to contacting the location.

In embodiments, the sporicidal solution comprises hydrogen peroxide and optionally comprises a silver stabilised hydrogen peroxide.

In embodiments, the sporicidal solution comprises chlorhexidine gluconate and about 70% isopropyl alcohol complex.

In an aspect of the present disclosure, there is provided a combination of an aptamer capable of specifically binding to C. difficile and a sporicidal agent and/or a sporostatic agent. The combination may be for use in the killing and/or inactivation of C. difficile.

In embodiments, the combination comprises a plurality of aptamers, the plurality of aptamers comprising two or more aptamers as defined herein.

In embodiments, the plurality of aptamers comprises an aptamer comprising or consisting essentially of a nucleic acid sequence as set forth in SEQ. ID. NO. 1; an aptamer comprising or consisting essentially of a nucleic acid sequence as set forth in SEQ. ID. NO. 5 and an aptamer comprising or consisting essentially of a nucleic acid sequence as set forth in SEQ. ID. NO. 6.

In embodiments, the combination comprises a sporicidal agent which comprises peracetic acid and/or an agent capable of generating peracetic acid.

In an aspect of the disclosure, there is provided a composition comprising one or more aptamers as defined herein and a sporicidal agent and/or a sporostatic agent.

In embodiments, the composition comprises an aptamer being capable of specifically binding to C. difficile. In embodiments, the composition comprises a plurality of aptamers, each aptamer being capable of specifically binding to the same epitope of a C. difficile spore. In embodiments, the plurality of aptamers comprises aptamers capable of binding to different epitopes of a C. difficile spore.

In embodiments, the plurality of aptamers comprises an aptamer comprising, consisting essentially of, or consisting of a nucleic acid sequence as set forth in SEQ. ID. NO. 1; an aptamer comprising, consisting essentially of, or consisting of a nucleic acid sequence as set forth in SEQ. ID. NO. 5; and an aptamer comprising, consisting essentially of, or consisting of a nucleic acid sequence as set forth in SEQ. ID. NO. 6.

In embodiments, the sporicidal agent comprises peracetic acid and/or agent capable of generating peracetic acid.

### DETAILED DESCRIPTION

Further features of certain embodiments of the present disclosure are described below. The practice of embodiments of the present disclosure will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al, Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y. Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure.

Units, prefixes and symbols are denoted in their Systeme International de Unitese (SI) accepted form. Numeric ranges are inclusive of the numbers defining the range. Unless otherwise indicated, amino acid sequences are written left to right in amino to carboxy orientation and nucleic acid sequences are written left to right in 5′ to 3′ orientation.

As used herein, the articles “a” and “an” refer to one or more than one, e.g., to at least one, of the grammatical object of the article. The use of the words “a” or “an” when used in conjunction with the term “comprising” herein may mean “one,” “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, kits and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

Clostridium difficile bacteria are found throughout the environment e.g. in soil, air, water, food products and human and animal faeces. A small number of people carry C. difficile in their intestinal tract without showing any symptoms. However, in other subjects, infection from C. difficile can cause symptoms ranging from diarrhea to life-threatening inflammation of the colon. Complications of C. difficile infection can include dehydration, kidney failure, toxic megacolon, perforation of the bowel and even death if the infection is not controlled quickly.

It commonly affects older adults in hospitals or long-term care facilities and subjects who have taken antibiotics and those with a compromised immune system are at greater risk of contracting C. difficile as are those who have undergone abdominal or gastrointestinal surgery. For example, the mortality rate of C. difficile infection can be up to 25% in frail, elderly people in hospitals. It has been postulated that the antibiotic therapy disrupts normal gut microbiota, allowing C. difficile colonization and growth because it is naturally resistant to many drugs used to treat other infections, thereby enabling its toxin production.

An increase of C. difficile infections in subjects previously considered to be low-risk such as younger and otherwise healthy individuals without exposure to health care facilities has been also been seen in recent years. A new strain of C. difficile, Type 027, has recently been identified. Type 027 is a strain that produces more toxins than most other types of C. difficile and it causes a greater proportion of severe disease and appears to have a higher mortality.

Metronidazole (400 mg or 500 mg, 3 times daily for 10-14 days) is considered in the UK to be the first-line for treating first episodes of mild to moderate C. difficile infection. Vancomycin (125 mg 4 times daily for 10-14 days) is an option for second episodes or if the infection is severe. An infection is defined as severe when there is a raised temperature or white cell count, rising creatinine or signs or symptoms of severe colitis. Vancomycin may also be used in infections caused by the type 027 strain. If infection recurs, vancomycin or fidaxomicin (200 mg twice daily for 10 days) may be used. In some severe cases, a person might have to have surgery to remove the infected part of the intestines.

Spores from C. difficile are passed in faeces and can be transmitted to food, surfaces and objects via unwashed hands. The spores can persist for weeks or months on surfaces and transmitted via contact with such surfaces.

Given the rise in antibiotic resistance and the potential mortality associated with C. difficile infection, control measures are of the highest importance. Current measures include healthcare providers such as nurses and doctors following protocols such as:
Clean their hands with soap and water or an alcohol-based hand rub before and after caring for every patient to prevent C. difficile and other germs from being passed from one patient to another on their hands.
Carefully clean hospital rooms and medical equipment that have been used for patients with C. difficile infection.
Only give patients antibiotics when it is necessary.
Use Contact Precautions to prevent C. difficile from spreading to other patients. Contact Precautions mean:
   Whenever possible, patients with C. difficile will have a single room or share a room only with someone else who also has C. difficile.
   Healthcare providers will put on gloves and wear a gown over their clothing while taking care of patients with C. difficile.
   Visitors may also be asked to wear a gown and gloves.
   When leaving the room, hospital providers and visitors remove their gown and gloves and clean their hands.
   Patients on Contact Precautions are asked to stay in their hospital rooms as much as possible. They can go to other areas of the hospital for treatments and tests.

Despite these preventative measures, C. difficile remains a significant healthcare issue and therefore there is a need for rapid identification of the presence of C. difficile in an environment to enable

It is an aim of some embodiments of the present disclosure to at least partially mitigate some of the problems identified in the prior art.

It is an aim of certain embodiments of the present disclosure to provide methods and products which have utility in the detection of C. difficile.

Clostridioides difficile (Formerly Clostridium difficile)

Aspects of the disclosure utilise aptamers which are capable of specifically binding to Clostridioides difficile (previously referred to as Clostridium difficile).

In some embodiments, the aptamer specifically binds to a target as defined herein. The term “target” as used herein is used to relate to a molecule selected from at least one of a C. difficile surface protein. In some embodiments, the target molecule is a target protein. In some embodiments, the term “target” as used herein is used to relate to a molecule selected from at least one of a C. difficile CotA protein, C. difficile CotE protein, C. difficile CdeC protein, C. difficile CdeM protein, C. difficile CotEC chitinase protein, and a C. difficile spore. As used herein, the terms “target protein” and “target peptide” are used interchangeably.

Certain embodiments of the present disclosure relate to aptamers which bind to a C. difficile spore. In some embodiments, the aptamer is selected against a whole C. difficile spore. Thus, in some embodiments, the aptamer selectively binds to a C. difficile spore.

Certain embodiments provide an aptamer which binds to a C. difficile spore coat protein. Embodiments of the present disclosure may utilise a plurality of aptamers, each aptamer being capable of binding to a unique site on the surface of a C. difficile spore. In some embodiments, the aptamer specifically binds to a surface protein of the exosporium layer of the C. difficile spore (e.g. CdeC, CdeM). In some embodiments, the aptamer specifically binds to a coat protein of the C. difficile spore (e.g. CotA, CotE, CotEC). In some embodiments the

In certain embodiments, the aptamer specifically binds to a protein as listed in Table 3 below. In embodiments, the disclosure comprises the use of a plurality of aptamers, each aptamer being capable of specifically bind to a protein listed in Table 3.

TABLE 3

| | |
|---|---|
| CotA | SEQ. ID. No. 15 |
| Cot E | SEQ. ID. No. 16 |
| CotEC | SEQ. ID. No. 17 |
| CdeC | SEQ. ID. No. 18 |
| CdeM | SEQ. ID. No. 19 |

### Target Proteins

C. difficile produces metabolically dormant spores. The spores comprise an outermost exosporium layer which may comprise a number of surface proteins. In embodiments, the exosporium layer comprises one or more proteins selected from BclA1, BclA2, BclA3, CdeA, CdeB, CdeC and CdeM.

### CdeC Protein

In embodiments, the aptamer specifically binds to a C. difficile CdeC protein. The amino acid sequence of CdeC is published under UniProtKB—Q18AS2 (Q18AS2_PEPD6) version 1 and is as set forth in SEQ ID NO. 18.

In embodiments, the aptamer binds to an epitope of the CdeC protein which is conserved between C. difficile strains. Thus, in certain embodiments, the aptamer is used to detect a plurality of C. difficile strains in a sample.

### CdeM Protein

In embodiments, the aptamer selectively binds to an amino acid sequence of a C. difficile surface-bound CdeM protein. CdeM is a cysteine rich protein which is understood to be required for the morphogenesis of the coat and exosporium layer of spores. An amino acid sequence of a C. difficile protein is published under UniProtKB—A0A3T1GTU1 (AOA3T1GTU1_CLODI) (version 1) and shown in SEQ ID NO: 19.

In embodiments, the aptamer binds to an epitope of the CdeM protein which is conserved between C. difficile strains. Thus, in certain embodiments, the aptamer is used to detect a plurality of C. difficile strains in a sample.

In embodiments, the spores comprise a spore coat. The spore coat may comprise a plurality of proteins including for example CotA and CotB.

### CotA

In embodiments, the aptamer specifically binds to a protein encoded by a C. difficile CotA gene. The protein may be referred to herein as either CotA or “spore coat assembly protein”.

An amino acid sequence of CotA is published under UniProtKB Accession No. Q186G8 (Q186G8_PEPD6) version 1 and shown in SEQ ID NO: 15.

### CotE

In embodiments, the aptamer specifically binds to a C. difficile protein encoded by a CotE gene. An amino acid sequence of a CotE protein (also referred to as peroxiredoxin) is published under accession number UniProtKB—Q18BV5 (Q18BV5_PEPD6) and is shown in SEQ ID NO: 16.

### CotEC Chitinase

In embodiments, the aptamer specifically binds to a recombinant C. difficile protein referred to as “CotEC” or “rCotE”. The amino acid sequence of CotEC consists of amino acid residues N281-F712 (SEQ ID NO: 20). The recombinant protein comprises a chitinase domain and a sequence unique to CotE. In some embodiments, the aptamer specifically binds to a recombinant C. difficile protein referred to as “rCotEC” (also referred to as AB45). The amino acid sequence of rCotEC consists of amino acid residues N381-F712 (SEQ ID NO: 17).

In embodiments, the aptamers are selected against a His-tagged CotEC protein. In some embodiments, the aptamers are selected against a tagged rCotEC protein, including but not limited to His-tagged rCotEC protein.

TABLE 2

| Target Protein | SEQ. ID. No |
|---|---|
| CotA | 15 |
| Cot E | 16 |
| CotEC | 17 |
| CdeC | 18 |
| CdeM | 19 |
| rCotE (LS25) | 20 |

C. difficile Spores

In embodiments, the aptamer is selected against a whole C. difficile spore. Thus, in certain embodiments, the aptamer selectively binds to a C. difficile spore. The C. difficile spore may be from a strain selected SH11, PCR ribotype 027, PCR ribotype 010, PCR ribotype 014 and ATCC® 43598™. In embodiments, the C. difficile may be a strain of a Clade selected from Clade 1, 2, 3, 4 and 5. In certain embodiments, the strain is selected from SH7 (Clade 1), SH8 (Clade 2), R20291 (Clade 2), SH9 (Clade 3), SH10 (Clade 4), ATCC 43598 (Clade 4) and SH11 (Clade 5). In embodiments, the strain is selected from R20291, ATCC 43598 and SH11.

In embodiments, the method comprises facilitating the killing and/or inactivation of a C. difficile strain selected from:

| Clade | Strain Name | Ribotype | Toxin A & B | Toxinotype |
|---|---|---|---|---|
| 1 | SH7 | RT015 | A+ | 0 |
| | | | B+ | |
| 2 | SH8 | RT176 | A+ | III |
| | | | B+ | |
| 3 | SH9 | RT023 | A+ | IV |
| | | | B+ | |
| 4 | SH10 | RT017 | A− | VIII |
| | | | B+ | |
| | ATCC43598 | RT017 | A− | VIII |
| | | | B+ | |
| 5 | SH11 | RT078 | A+ | V |
| | | | B+ | |

In certain embodiments, there is provided an aptamer comprising a nucleic acid sequence selected from a nucleic acid sequence as set forth in Table 1 or Table 4.

TABLE 1
Aptamer Sequences
Sequence

CCAGTGTAGACTACTCAATGCTCTTACGATCCTCACCTGCTAGCACACCC
ATATCCCATGCGTACTATCCACAGGTCAACC (SEQ ID NO: 1)
CCAGTGTAGACTACTCAATGCGGGTTGCGACATGGTGGTAAGAGCTCAGC
CCGTTCCCATAGTACTATCCACAGGTCAACC (SEQ ID NO: 2)
CCAGTGTAGACTACTCAATGCACGGCCTGTTCGTAAGACCCTTACAGACT
AGTTTTTCCCTGTACTATCCACAGGTCAACC (SEQ ID NO: 3)
CCAGTGTAGACTACTCAATGCCCTATTAGCTGTATCGATCCGTTTAGTCG
CTCCTCCGATAGTACTATCCACAGGTCAACC (SEQ ID NO: 4)
CCAGTGTAGACTACTCAATGCCTGGTAAATCGATGACCGCTGCCTCGCCT
GAGTAATCATCGTACTATCCACAGGTCAACC (SEQ ID NO: 5)
CCAGTGTAGACTACTCAATGCCGTGGACTGGTCGGGTTTGGATTCGGCAG
ATGAATCAGTAGTACTATCCACAGGTCAACC (SEQ ID NO: 6)
CCAGTGTAGACTACTCAATGCCTTGTAAGAAGAACAATCGCCGCTTCGCC
TGAATAGGTTCGTACTATCCACAGGTCAACC (SEQ ID NO: 7)
CCAGTGTAGACTACTCAATGCGGACCGTTGCCTCGCCCGAGTAATCCGCC
ATCGCCTTTCCGTACTATCCACAGGTCAACC (SEQ ID NO: 8)
CCAGTGTAGACTACTCAATGCTTAAGTTCTGGGGACACGTGATGAACGCA
TTTAATGGGGCGTACTATCCACAGGTCAACC (SEQ ID NO: 9)
CCAGTGTAGACTACTCAATGCCGTGGACTGGTCGGGTTTGGATTCGGCAG
ATGAATCACTAGTACTATCCACAGGTCAACC (SEQ ID NO: 10)
CCAGTGTAGACTACTCAATGCGGCTGTGTGACTTGACCTTTGGAATGGGT
GGGAGGGATGGGTACTATCCACAGGTCAACC (SEQ ID NO: 11)
CCAGTGTAGACTACTCAATGCGGTGTGGTGACCTTGACCTATGGAACCTG
GTTGTAGTACTATCCACAGGTCAACC (SEQ ID NO: 12)
CCAGTGTAGACTACTCAATGCTCGACATTTCCGCCCCGACGGCCCTCCTA
GTGATGGGGAGAGTACTATCCACAGGTCAACC (SEQ ID NO: 13)
CCAGTGTAGACTACTCAATGCCTTCCATTCACCTACCGAGCTAAGCGTTC
GACTTAGGTCTGTACTATCCACAGGTCAACC (SEQ ID NO: 14)
ATCGATGACCGCTGCCTCGCCTGAGTAATCATCGTA (SEQ ID NO:
23)
CCATACTCAATGCTCTTACGATCCTCATCAACC (SEQ ID NO: 24)
CCAGTGTAGACTACTCAATGCTCTTACGATCCTCATCAACC (SEQ ID
NO: 25)
AGTGTAGACTACTCAATGCGGCTGGCCACAGGTCAACC (SEQ ID NO:
26)
CTTGACCTTTGGAATGGGTGGGAGGGATGGGTACTATCCACAGGTCAACC
(SEQ ID NO: 27)
AATGGGTGGGAGGGATGGGTACTA (SEQ ID NO: 28)
CTTGACCTTTGGAATGGGTAGGGAGGGAGGGATACTATCCACAGGTCAAC
C (SEQ ID NO: 29)
CTTGACCTTTGGAATGGGTGGGAGGGAGGGTATCCACAGGTCAACC
(SEQ ID NO: 30)
ACTACTCAATGCTCGACATTTCCGCCCCGACGGCCCTCCTAGTGAGGGGA
GAGTAGA (SEQ ID NO: 31)
ACTACTCAATGCTCGACATTTCCGCCCCGACGGCCCTCCTAGTGATGGGG
AGAGTAGA (SEQ ID NO: 32)
ACTCAAGGCCGTGGACTGGTCGGGTTTGGATTCGGCAGATGAATCACT
(SEQ ID NO: 33)
ACTCAAGGCCGTGGACTGGTCGGGTTTGGAT (SEQ ID NO: 34)
ACCCGTGGGACTGGGTCGGGTCGGG (SEQ ID NO: 35)
AACTGCCTGGTAAATCGATGACCGCTGCCTCGCCTGAGTAATCATCGTAC
TATCCACAGGTC (SEQ ID NO: 36)
GTAAATCGATGACCGCTGCCTCGCCTGAGTAATCATCGTAC (SEQ ID
NO: 37)
ACTACTCAAACCCGTGGACTGGTCGGGTTTGGATTCGGCAGATGAATCAG
TAGAAA (SEQ ID NO: 38)
ACTACTCAATGCCGTGGACTGGTCGGGTTTGGAATCGGCAGATGAATCAG
TAGTAAA (SEQ ID NO: 39)
CTCAATGCCTTCCATTCACCTACCGAGCTAAGCGTTCGACTTAGGTCTGT
ACT (SEQ ID NO: 42)
CCTACCGAGCTAAGCGTTCGACTTAGGTCTGTACT (SEQ ID NO:
43)
CTCACCTGCTAGCACACCCATATCCCATGGGTACAATCCACAGGTCAA
(SEQ ID NO: 44)
CTCACCTGCTAGCACACCCACATCCCGTGCGTGCTATCCACAGGTGAA
(SEQ ID NO: 45)
AGTGCAGACTACTCAATGCACGGCCGGTTCGGAAGACCCTTCCAGACTAG
TTTTTCCCTGTACTAGTCCACCGGCTA (SEQ ID NO: 46)
AGTGCAGACTACTCAATGCACGGCCTGGTTCGTAAGACCCTTACCAGACT
(SEQ ID NO: 47)
CGGTTGCGACATGGTGGTAAGAGCTCAGCCCGTTCCCATAGTACTATCCA
CAGGTCAACCT (SEQ ID NO: 48)
CGGTTGCGACATGGTGGTAAGAGCTCAGCCCGTTCCCATAGTACTATCCA
CAGGTCGCAACCT (SEQ ID NO: 49)
CCCGTGTAGACTACTCAATGCGGGCTGCGACATGGTGGTAAGAGCTCAGC
CCGTTCCCATAGTACTATCCACGGGT (SEQ ID NO: 50)
CCCGTGTAGACTATTTTAGTACTATCCACGGG (SEQ ID NO: 51)
TGCGGGCTGCGACATGGTGGTAAGAGCTCAGCCCGTT (SEQ ID NO:
52)
ACCCAGGTGTAGGACGACTCAATGCCCTATTAGCTGTATCGATCCGTTTA
GTCGCTCCTCCGATAGTACCCTATCCACCAGGGA (SEQ ID NO: 53)
ACCAGGTGGTAGACCTACTCACATGCCCTATTAGCGTGTATCGATCCGGT
TTAGTCCGCTTCGATAGTAGUCCCACCAGGA (SEQ ID NO: 54)
CTCAATGCCTTCCATTCACCTACCGAGCTAAGCGTTCGACTTAGGTCTGT
ACT (SEQ ID NO: 55)

TABLE 4
Primer regions are indicated in bold and italic

| ID | Sequence | Target |
|---|---|---|
| C.diff_F1 | CCAGTGTAGACTACTCAATGCTCTTACGATCCT | C.diff spores |
| | CACCTGCTAGCACACCCATATCCCATGCGTACT | |
| | ATCCACAGGTCAACC (SEQ ID NO: 1) | |
| C.diff_G1 | CCAGTGTAGACTACTCAATGCGGGTTGCGACA | C.diff spores |
| | TGGTGGTAAGAGCTCAGCCCGTTCCCATAGTAC | |
| | TATCCACAGGTCAACC (SEQ ID NO: 2) | |
| C.diff_E2 | CCAGTGTAGACTACTCAATGCACGGCCTGTTCG | C.diff spores |
| | TAAGACCCTTACAGACTAGTTTTTCCCTGTACTA | |
| | TCCACAGGTCAACC (SEQ ID NO: 3) | |
| Chitinase_D10 | CCAGTGTAGACTACTCAATGCCCTATTAGCTGT | CotEC |
| | ATCGATCCGTTTAGTCGCTCCTCCGATAGTACTA | Chitinase |
| | TCCACAGGTCAACC (SEQ ID NO: 4) | |
| Chitinase_D11 | CCAGTGTAGACTACTCAATGCCTGGTAAATCGA | CotEC |
| | TGACCGCTGCCTCGCCTGAGTAATCATCGTACT | Chitinase |
| | ATCCACAGGTCAACC (SEQ ID NO: 5) | |
| CdeC_D1 | CCAGTGTAGACTACTCAATGCCGTGGACTGGT | CdeC |
| | CGGGTTTGGATTCGGCAGATGAATCAGTAGTAC | |
| | TATCCACAGGTCAACC (SEQ ID NO: 6) | |
| Chitinase_H11 | CCAGTGTAGACTACTCAATGCCTTGTAAGAAGA | CotEC |
| | ACAATCGCCGCTTCGCCTGAATAGGTTCGTACT | Chitinase |
| | ATCCACAGGTCAACC (SEQ ID NO: 7) | |
| Chitinase_D7 | CCAGTGTAGACTACTCAATGCGGACCGTTGCCT | CotEC |
| | CGCCCGAGTAATCCGCCATCGCCTTTCCGTACT | Chitinase |
| | ATCCACAGGTCAACC (SEQ ID NO: 8) | |
| CotA_B1 | CCAGTGTAGACTACTCAATGCTTAAGTTCTGGG | CotA |
| | GACACGTGATGAACGCATTTAATGGGGCGTACT | |
| | ATCCACAGGTCAACC (SEQ ID NO: 9) | |
| CotA_C1 | CCAGTGTAGACTACTCAATGCCGTGGACTGGT | CotA |
| | CGGGTTTGGATTCGGCAGATGAATCACTAGTAC | |
| | TATCCACAGGTCAACC (SEQ ID NO: 10) | |
| CotE_H2 | CCAGTGTAGACTACTCAATGCGGCTGTGTGACT | CotE |
| | TGACCTTTGGAATGGGTGGGAGGGATGGGTACT | |
| | ATCCACAGGTCAACC (SEQ ID NO: 11) | |
| CotE_E2 | CCAGTGTAGACTACTCAATGCGGTGTGGTGAC | CotE |
| | CTTGACCTATGGAACCTGGTTGTAGTACTATCC | |
| | ACAGGTCAACC (SEQ ID NO: 12) | |
| CotE_D2 | CCAGTGTAGACTACTCAATGCTCGACATTTCCG | CotE |
| | CCCCGACGGCCCTCCTAGTGATGGGGAGAGTAC | |
| | TATCCACAGGTCAACC (SEQ ID NO: 13) | |
| CdeM_E2 | CCAGTGTAGACTACTCAATGCCTTCCATTCACC | CdeM |
| | TACCGAGCTAAGCGTTCGACTTAGGTCTGTACT | |
| | ATCCACAGGTCAACC (SEQ ID NO: 14) | |
| Chitinase_D11 | ATCGATGACCGCTGCCTCGCCTGAGTAATCATCG | CotEC |
| | TA (SEQ ID NO: 23) | Chitinase |
| C.diff_F1 | CCATACTCAATGCTCTTACGATCCTCATCAACC | C.diff spores |
| | (SEQ ID NO: 24) | |
| C.diff_G1 | CCAGTGTAGACTACTCAATGCTCTTACGATCCTC | C.diff spores |
| | ATCAACC (SEQ ID NO: 25) | |
| CotE_H2 | AGTGTAGACTACTCAATGCGGCTGGCCACAGGT | CotE |
| | CAACC (SEQ ID NO: 26) | |
| CotE_H2 | CTTGACCTTTGGAATGGGTGGGAGGGATGGGTA | CotE |
| | CTATCCACAGGTCAACC (SEQ ID NO: 27) | |
| CotE_H2 | AATGGGTGGGAGGGATGGGTACTA (SEQ ID | CotE |
| | NO: 28) | |
| CotE_H2 | CTTGACCTTTGGAATGGGTAGGGAGGGAGGGAT | CotE |
| | ACTATCCACAGGTCAACC (SEQ ID NO: 29) | |
| CotE_H2 | CTTGACCTTTGGAATGGGTGGGAGGGAGGGTAT | CotE |
| | CCACAGGTCAACC (SEQ ID NO: 30) | |
| CotE_D2 | ACTACTCAATGCTCGACATTTCCGCCCCGACGGC | CotE |
| | CCTCCTAGTGAGGGGAGAGTAGA (SEQ ID | |
| | NO: 31) | |
| CotE_D2 | ACTACTCAATGCTCGACATTTCCGCCCCGACGGC | CotE |
| | CCTCCTAGTGATGGGGAGAGTAGA (SEQ ID | |
| | NO: 32) | |
| CotA_C1 | ACTCAAGGCCGTGGACTGGTCGGGTTTGGATTC | CotA |
| | GGCAGATGAATCACT (SEQ ID NO: 33) | |
| CotA_C1 | ACTCAAGGCCGTGGACTGGTCGGGTTTGGAT | CotA |
| | (SEQ ID NO: 34) | |
| CotA_C1 | ACCCGTGGGACTGGGTCGGGTCGGG (SEQ ID | CotA |
| | NO: 35) | |
| Chitinase_D11 | AACTGCCTGGTAAATCGATGACCGCTGCCTCGC | CotEC |
| | CTGAGTAATCATCGTACTATCCACAGGTC | Chitinase |
| | (SEQ ID NO: 36) | |
| Chitinase_D11 | GTAAATCGATGACCGCTGCCTCGCCTGAGTAATC | CotEC |
| | ATOGTAC (SEQ ID NO: 37) | Chitinase |
| CdeC_D1 | ACTACTCAAACCCGTGGACTGGTCGGGTTTGGA | CdeC |
| | TTCGGCAGATGAATCAGTAGAAA (SEQ ID | |
| | NO: 38) | |
| CdeC_D1 | ACTACTCAATGCCGTGGACTGGTCGGGTTTGGA | CdeC |
| | ATCGGCAGATGAATCAGTAGTAAA (SEQ ID | |
| | NO: 39) | |
| CdeM_E2 | CCTACCGAGCTAAGCGTTCGACTTAGGTCTGTAC | CdeM |
| | T (SEQ ID NO: 43) | |
| C.diff_F1 | CTCACCTGCTAGCACACCCATATCCCATGGGTAC | C.diff spores |
| | AATCCACAGGTCAA (SEQ ID NO: 44) | |
| C.diff_F1 | CTCACCTGCTAGCACACCCACATCCCGTGCGTGC | C.diff spores |
| | TATCCACAGGTGAA (SEQ ID NO: 45) | |
| C.diff_E2 | AGTGCAGACTACTCAATGCACGGCCGGTTCGGAA | C.diff spores |
| | GACCCTTCCAGACTAGTTTTTCCCTGTACTAGT | |
| | CCACCGGCTA (SEQ ID NO: 46) | |
| C.diff_E2 | AGTGCAGACTACTCAATGCACGGCCTGGTTCGT | C.diff spores |
| | AAGACCCTTACCAGACT (SEQ ID NO: 47) | |
| C.diff_G1 | CGGTTGCGACATGGTGGTAAGAGCTCAGCCCGT | C.diff spores |
| | TCCCATAGTACTATCCACAGGTCAACCT (SEQ | |
| | ID NO: 48) | |
| C.diff_G1 | CGGTTGCGACATGGTGGTAAGAGCTCAGCCCGT | C.diff spores |
| | TCCCATAGTACTATCCACAGGTCGCAACCT | |
| | (SEQ ID NO: 49) | |
| C.diff_G1 | CCCGTGTAGACTACTCAATGCGGGCTGCGACAT | C.diff spores |
| | GGTGGTAAGAGCTCAGCCCGTTCCCATAGTACT | |
| | ATCCACGGGT (SEQ ID NO: 50) | |
| C.diff_G1 | CCCGTGTAGACTATTTTAGTACTATCCACGGG | C.diff spores |
| | (SEQ ID NO: 51) | |
| C.diff_G1 | TGCGGGCTGCGACATGGTGGTAAGAGCTCAGCC | C.diff spores |
| | CGTT (SEQ ID NO: 52) | |
| Chitinase_D10 | ACCCAGGTGTAGGACGACTCAATGCCCTATTAGC | CotEC |
| | TGTATCGATCCGTTTAGTCGCTCCTCCGATAGTA | Chitinase |
| | CCCTATCCACCAGGGA (SEQ ID NO: 53) | |
| Chitinase_D10 | ACCAGGTGGTAGACCTACTCACATGCCCTATTAG | CotEC |
| | CGTGTATCGATCCGGTTTAGTCCGCTTCGATAGT | Chitinase |
| | AGUCCCACCAGGA (SEQ ID NO: 54) | |
| CdeM_E2 | CTCAATGCCTTCCATTCACCTACCGAGCTAAGCG | CdeM |
| | TTCGACTTAGGTCTGTACT (SEQ ID NO: 55) | |

In an embodiment, the aptamer specifically binds to a target as defined herein. The term “target” as used herein is used to relate to molecule selected from a C. difficile CotA protein, C. difficile CotE protein, C. difficile CdeC protein, C. difficile CdeM protein, C. difficile CotEC chitinase protein and a C. difficile spore.

In an embodiment, the aptamer specifically binds to an epitope in a C. difficile CotA protein.

In an embodiment, the aptamer specifically binds to an epitope in a C. difficile CotE protein.

In an embodiment, the aptamer specifically binds to an epitope in a C. difficile CdeC protein.

In an embodiment, the aptamer specifically binds to an epitope in a C. difficile CdeM protein.

In an embodiment, the aptamer specifically binds to an epitope in a C. difficile CotEC chitinase protein.

In an embodiment, the aptamer specifically binds to an epitope on a surface of a C. difficile spore.

An aptamer binds “specifically” to a target as defined herein if the aptamer binds with preferential or high affinity to the target, but does not bind or binds with only low affinity to other structurally related molecules (e.g. Bacillus subtilis spores.) In some embodiments, the dissociation constant for the target protein is in the micro-molar range. In some embodiments, the dissociation constant for the target protein is in the nano-molar (nM) range. In some embodiments, the dissociation constant for the target protein is in the pico-molar (pM) range. In some embodiments, the dissociation constant is about 0.1 nM or less. In some embodiments, the dissociation constant is about 0.1 nM to about 1 nM. In some embodiments, the dissociation constant is about 1 nM to about 10 nM. In some embodiments, the dissociation constant is about 10 nM to about 100 nM. In some embodiments, the dissociation constant is about 100 nM to about 1000 nM. Lower affinity binding may refer to binding that occurs at less affinity than to a target protein. Lower affinity binding may refer to binding that occurs at less affinity than to a target. The lower affinity binding may be selected from the range of less than 1 fold to 2 fold, less than 2 fold to 5 fold, less than 5 fold to 10 fold, less than 10 fold to 50 fold, less than 100 fold to 1000 fold, less than 1000 fold to 10000 fold, or less than 10000 fold to 100000 fold of binding to the target.

### Aptamers

The aptamers described herein are small artificial ligands, compromising DNA, RNA or modifications thereof, capable of specifically binding to a target as defined herein with high affinity and specificity.

As used herein, “aptamer”, “nucleic acid molecule” or “oligonucleotide” are used interchangeably to refer to a non-naturally occurring nucleic acid molecule that has a desirable action on a target as defined herein.

The aptamers of the disclosure may be DNA aptamers. For example, the aptamers may be formed from single-stranded DNA (ssDNA). Alternatively, the aptamers of the disclosure may be RNA aptamers. For example, the aptamers can be formed from single-stranded RNA (ssRNA).

In certain embodiments, the aptamers are RNA aptamers and comprise a sequence in which of one or some or all of the deoxyribonucleotides in any of the sequences set forth in SEQ. ID NOs. 1 to 14, 23 to 39, 42 to 55 are substituted for their equivalent ribonucleotide residues AMP, GMP, UMP or CMP.

The aptamers of the disclosure may comprise modified nucleic acids as described herein.

In certain embodiments, the aptamers of the disclosure are prepared using principles of in vitro selection known in the art, that include iterative cycles of target binding, partitioning and preferential amplification of target binding sequences. Selection may be performed using immobilized target proteins. Immobilization may include, but is not limited to, immobilization to a solid surface. In a non-limiting example, the solid surface may be beads. In a non-limiting example, the solid surface may be magnetic beads.

Non-limiting examples of amplification methods include polymerase chain reaction (PCR), ligation amplification (or ligase chain reaction, LCR), strand displacement amplification, nucleic acid sequence-based amplification, and amplification methods based on the use of Q-beta replicase. In a non-limiting embodiment, at least one type of aptamer may be immobilized on a solid surface during amplification. Each of these exemplary methods is well known in the art.

In embodiments, the aptamers are selected from a nucleic acid molecule library such as a single-stranded DNA or RNA nucleic acid molecule library. Typically, the aptamers are selected from a “universal aptamer selection library” that is designed such that any selected aptamers need little to no adaptation to convert into any of the listed assay formats.

Once selected, the aptamer may be further modified before being used e.g. to remove one or both primer sequences and/or parts of the randomised not required for target binding.

Typically, aptamers for use in the disclosure comprise a first primer region (e.g. at the 5′ end), a second primer region (e.g. at the 3′ end), or both. The primer regions may serve as primer binding sites for PCR amplification of the library and selected aptamers.

The skilled person would understand different primer sequences can be selected depending, for example, on the starting library and/or aptamer selection protocol. In an embodiment, aptamers may comprise SEQ ID NO: 21 and/or 22. In other embodiments, any one of one to all of the nucleotides disclosed by SEQ ID NO: 21 or 22 may be modified. The primer region length may also be varied.

The first primer region and/or second region may comprise a detectable label as described herein. In an embodiment, the first and/or second primer region may be fluorescently labelled. Non-limiting examples of fluorescent labels include but are not limited to fluorescein, green fluorescent protein (GFP), yellow fluorescent protein, cyan fluorescent protein, and others. In an embodiment, a fluorescein label is used. In embodiments, other forms of detecting the primer may be used, including but not limited to phosphate (PO₄) labelling, isotope labelling, electrochemical sensors, colorimetric biosensors, and others.

In embodiments, the aptamers of the disclosure comprise or consist of a nucleic acid sequence selected from any one of SEQ ID NOs: 1 to 14, 23 to 39 or 43 to 55.

In certain embodiments, aptamers of the disclosure comprise or consist of a nucleic acid sequence having at least 85%, at least 90%, at least 95%, at least 96%, at least 97%, at least 98%, at least 99% or more sequence identity to the nucleotide sequence of any one of SEQ ID NOs: 1 to 14, 23 to 39 or 43 to 55.

As used herein, “sequence identity” refers to the percentage of nucleotides in a candidate sequence that are identical with the nucleotides in said sequences after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, CLUSTALW or Megalign (DNASTAR) software. For example, % nucleic acid sequence identity values can be generated using sequence comparison computer programs found on the European Bioinformatics Institute website (www.ebi.ac.uk).

As used herein, when describing the percent identity of a nucleic acid, such as an aptamer, the sequence of which is at least, for example, about 90% identical to a reference nucleotide sequence, it is intended that the nucleic acid sequence is identical to the reference sequence except that the nucleic acid sequence may include up to ten-point mutations (e.g. substitution, deletion, insertion) per each 100 nucleotides of the reference nucleic acid sequence. These mutations may occur at the 5′ or 3′ terminal positions of the reference nucleotide sequence or anywhere between those 5′ or 3′ terminal positions, interspersed either individually among nucleotides in the reference sequence or in one or more contiguous groups within the reference sequence.

In embodiments, aptamers comprise, consist essentially of, or consist of a minimal effective fragment of SEQ ID NOs: 1 to 14, 23 to 39 or 43 to 55. Herein, a “minimal effective fragment” is understood to mean a fragment (e.g. portion) of the full-length aptamer capable of binding to a target as defined herewith with the same or improved affinity as compared to the full-length aptamer. A minimal effective fragment may compete for binding to a target as defined herein with the full-length aptamer.

In embodiments, the aptamers of the disclosure comprise, consist essentially of, or consist of at least 10 contiguous nucleic acid residues of any of the sequences as set forth in any one of SEQ ID NOs: 1 to 14, 23 to 39 or 43 to 55 and show equivalent or improved binding to the target molecule. In embodiments, the aptamers of the disclosure comprise, consist essentially of, or consist of at least 10 contiguous nucleic acid residues of any of the sequences as set forth in any one of SEQ ID NOs: 1 to 14, 23 to 39 or 43 to 55 and show adequate binding to the target molecule. Adequate binding includes binding to target molecule that occurs with affinity and specificity as described herein, or an affinity and/or specificity of binding less than that of the full-length aptamer sequence above but still capable of delivering a report of the presence of its respective target.

In embodiments, the aptamers of the disclosure comprise, consist essentially of, or consist of at least 20 contiguous nucleic acid residues of any of the sequences as set forth in any one of SEQ ID NOs: 1 to 14, 23 to 39 or 43 to 55 and show equivalent or improved binding to the target molecule. In embodiments, the aptamers of the disclosure comprise, consist essentially of, or consist of at least 20 contiguous nucleic acid residues of any of the sequences as set forth in any one of SEQ ID NOs: 1 to 14, 23 to 39 or 43 to 55 and show adequate binding to the target molecule. Adequate binding includes binding to target molecule that occurs with affinity and specificity as described herein, or an affinity and/or specificity of binding less than that of the full-length aptamer sequence above but still capable of delivering a report of the presence of its respective target.

In embodiments, an aptamer of the disclosure comprise, consists essentially of, or consist of at least 24 contiguous nucleotides of any of the sequences as set forth in any one of SEQ ID NOs: 1 to 14, 23 to 39 or 43 to 55.

In some embodiments, an aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 1. The aptamer may comprise, consist essentially of, or consist of any span of contiguous nucleotides from SEQ ID NO: 1, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 2. The aptamer may comprise, consist essentially of, or consist of any span of contiguous nucleotides from SEQ ID NO: 2, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 3. The aptamer may comprise, consists essentially of, or consist of any span of contiguous nucleotides from SEQ ID NO: 3, where the span has a length chosen in one nucleotide increments from 25 nucleotides to full length. The aptamer may comprise, consists essentially of, or consist of any span of contiguous nucleotides from SEQ ID NO: 3, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 4. The aptamer may comprise, consists essentially of, or consist of any span of contiguous nucleotides from SEQ ID NO: 4, where the span has a length chosen in one nucleotide increments from 25 nucleotides to full length. The aptamer may comprise, consists essentially of, or consist of any span of contiguous nucleotides from SEQ ID NO: 4, where the span has a length chosen in one nucleotide increments from 25 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 5. The aptamer may comprise, consists essentially of, or consist of any span of contiguous nucleotides from SEQ ID NO: 5, where the span has a length chosen in one nucleotide increments from 25 nucleotides to full length. The aptamer may comprise, consists essentially of, or consist of any span of contiguous nucleotides from SEQ ID NO: 5, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 6. The aptamer may comprise, consists essentially of, or consist of any span of contiguous nucleotides from SEQ ID NO: 6, where the span has a length chosen in one nucleotide increments from 25 nucleotides to full length. The aptamer may comprise, consists essentially of, or consist of any span of contiguous nucleotides from SEQ ID NO: 6, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleic acid molecules as comprised in the nucleic acid sequence of SEQ ID NO: 7. The aptamer may comprise, consists essentially of, or consist of any span of contiguous nucleotides from SEQ ID NO: 7, where the span has a length chosen in one nucleotide increments from 25 nucleotides to full length. The aptamer may comprise, consists essentially of, or consist of any span of contiguous nucleotides from SEQ ID NO: 7, where the span has a length chosen in one nucleotide increments from 10 nucleotides to full length.

In embodiments, the aptamer comprises, consists essentially of, or consists of 25, 26, 27, 28, 29 30, 31, 32, 33, 34, 35, 36, 37, 38, 39 or 40 contiguous nucleotides in the nucleic acid sequence of SEQ ID NO: 8. The aptamer may comprise, consists essentially of, or consist of any span of contiguous nucleotides from SEQ ID NO: 8, where the span has a length chosen in one nucleotide increments from 25 nucleotides to full length. The aptamer may comprise, consists essentially of, or consist of any span of contiguous nucleotides from SEQ ID NO: 8, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 9. The aptamer may comprise, consists essentially of, or consist of any span of contiguous nucleotides from SEQ ID NO: 9, where the span has a length chosen in one nucleotide increments from 25 nucleotides to full length. The aptamer may comprise, consists essentially of, or consist of any span of contiguous nucleotides from SEQ ID NO: 9, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 10. The aptamer may comprise, consists essentially of, or consist of any span of contiguous nucleotides from SEQ ID NO: 10, where the span has a length chosen in one nucleotide increments from 25 nucleotides to full length. The aptamer may comprise, consists essentially of, or consist of any span of contiguous nucleotides from SEQ ID NO: 10, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 11. The aptamer may comprise, consists essentially of, or consist of any span of contiguous nucleotides from SEQ ID NO: 11, where the span has a length chosen in one nucleotide increments from 25 nucleotides to full length. The aptamer may comprise, consists essentially of, or consist of any span of contiguous nucleotides from SEQ ID NO: 11, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76 contiguous nucleotides in the nucleic acid sequence of SEQ ID NO: 12. The aptamer may comprise, consists essentially of, or consist of any span of contiguous nucleotides from SEQ ID NO: 12, where the span has a length chosen in one nucleotide increments from 25 nucleotides to full length. The aptamer may comprise, consists essentially of, or consist of any span of contiguous nucleotides from SEQ ID NO: 12, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 nucleotides in the nucleic acid sequence of SEQ ID NO: 13. The aptamer may comprise, consists essentially of, or consist of any span of contiguous nucleotides from SEQ ID NO: 13, where the span has a length chosen in one nucleotide increments from 25 nucleotides to full length. The aptamer may comprise, consists essentially of, or consist of any span of contiguous nucleotides from SEQ ID NO: 13, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 14. The aptamer may comprise, consists essentially of, or consist of any span of contiguous nucleotides from SEQ ID NO: 14, where the span has a length chosen in one nucleotide increments from 14 nucleotides to full length. The aptamer may comprise, consists essentially of, or consist of any span of contiguous nucleotides from SEQ ID NO: 14, where the span has a length chosen in one nucleotide increments from 10 nucleotides to full length.

In embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, or 36 contiguous nucleotides in the nucleic acid sequence of SEQ ID NO: 23. The aptamer may comprise, consists essentially of, or consist of any span of contiguous nucleotides from SEQ ID NO: 23, where the span has a length chosen in one nucleotide increments from 25 nucleotides to full length. The aptamer may comprise, consists essentially of, or consist of any span of contiguous nucleotides from SEQ ID NO: 23, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32 or 33 contiguous nucleotides in the nucleic acid sequence of SEQ ID NO: 24. The aptamer may comprise, consists essentially of, or consist of any span of contiguous nucleotides from SEQ ID NO: 24, where the span has a length chosen in one nucleotide increments from 25 nucleotides to full length. The aptamer may comprise, consist essentially of, or consist of any span of contiguous nucleotides from SEQ ID NO: 24, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 contiguous nucleotides in the nucleic acid sequence of SEQ ID NO: 25. The aptamer may comprise, consists essentially of, or consist of any span of contiguous nucleotides from SEQ ID NO: 25, where the span has a length chosen in one nucleotide increments from 25 nucleotides to full length. The aptamer may comprise, consist essentially of, or consist of any span of contiguous nucleotides from SEQ ID NO: 25, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, or 38 contiguous nucleotides in the nucleic acid sequence of SEQ ID NO: 26. The aptamer may comprise, consists essentially of, or consist of any span of contiguous nucleotides from SEQ ID NO: 26, where the span has a length chosen in one nucleotide increments from 25 nucleotides to full length. The aptamer may comprise, consist essentially of, or consist of any span of contiguous nucleotides from SEQ ID NO: 26, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides in the nucleic acid sequence of SEQ ID NO: 27. The aptamer may comprise, consists essentially of, or consist of any span of contiguous nucleotides from SEQ ID NO: 27, where the span has a length chosen in one nucleotide increments from 25 nucleotides to full length. The aptamer may comprise, consist essentially of, or consist of any span of contiguous nucleotides from SEQ ID NO: 27, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, or 24 contiguous nucleotides in the nucleic acid sequence of SEQ ID NO: 28. The aptamer may comprise, consist essentially of, or consist of any span of contiguous nucleotides from SEQ ID NO: 28, where the span has a length chosen in one nucleotide increments from 15 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 contiguous nucleotides in the nucleic acid sequence of SEQ ID NO: 29. The aptamer may comprise, consists essentially of, or consist of any span of contiguous nucleotides from SEQ ID NO: 29, where the span has a length chosen in one nucleotide increments from 25 nucleotides to full length. The aptamer may comprise, consist essentially of, or consist of any span of contiguous nucleotides from SEQ ID NO: 29, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 contiguous nucleotides in the nucleic acid sequence of SEQ ID NO: 30. The aptamer may comprise, consists essentially of, or consist of any span of contiguous nucleotides from SEQ ID NO: 30, where the span has a length chosen in one nucleotide increments from 25 nucleotides to full length. The aptamer may comprise, consist essentially of, or consist of any span of contiguous nucleotides from SEQ ID NO: 30, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 contiguous nucleotides in the nucleic acid sequence of SEQ ID NO: 31. The aptamer may comprise, consists essentially of, or consist of any span of contiguous nucleotides from SEQ ID NO: 31, where the span has a length chosen in one nucleotide increments from 25 nucleotides to full length. The aptamer may comprise, consist essentially of, or consist of any span of contiguous nucleotides from SEQ ID NO: 31, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 contiguous nucleotides in the nucleic acid sequence of SEQ ID NO: 32. The aptamer may comprise, consists essentially of, or consist of any span of contiguous nucleotides from SEQ ID NO: 24, where the span has a length chosen in one nucleotide increments from 32 nucleotides to full length. The aptamer may comprise, consist essentially of, or consist of any span of contiguous nucleotides from SEQ ID NO: 32, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 contiguous nucleotides in the nucleic acid sequence of SEQ ID NO: 33. The aptamer may comprise, consists essentially of, or consist of any span of contiguous nucleotides from SEQ ID NO: 33, where the span has a length chosen in one nucleotide increments from 25 nucleotides to full length. The aptamer may comprise, consist essentially of, or consist of any span of contiguous nucleotides from SEQ ID NO: 33, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, or 31 contiguous nucleotides in the nucleic acid sequence of SEQ ID NO: 34. The aptamer may comprise, consists essentially of, or consist of any span of contiguous nucleotides from SEQ ID NO: 34, where the span has a length chosen in one nucleotide increments from 25 nucleotides to full length. The aptamer may comprise, consist essentially of, or consist of any span of contiguous nucleotides from SEQ ID NO: 34, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, or 25 contiguous nucleotides in the nucleic acid sequence of SEQ ID NO: 35. The aptamer may comprise, consist essentially of, or consist of any span of contiguous nucleotides from SEQ ID NO: 35, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 contiguous nucleotides in the nucleic acid sequence of SEQ ID NO: 36. The aptamer may comprise, consists essentially of, or consist of any span of contiguous nucleotides from SEQ ID NO: 36, where the span has a length chosen in one nucleotide increments from 25 nucleotides to full length. The aptamer may comprise, consist essentially of, or consist of any span of contiguous nucleotides from SEQ ID NO: 36, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 contiguous nucleotides in the nucleic acid sequence of SEQ ID NO: 37. The aptamer may comprise, consists essentially of, or consist of any span of contiguous nucleotides from SEQ ID NO: 37, where the span has a length chosen in one nucleotide increments from 25 nucleotides to full length. The aptamer may comprise, consist essentially of, or consist of any span of contiguous nucleotides from SEQ ID NO: 37, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 contiguous nucleotides in the nucleic acid sequence of SEQ ID NO: 38. The aptamer may comprise, consists essentially of, or consist of any span of contiguous nucleotides from SEQ ID NO: 38, where the span has a length chosen in one nucleotide increments from 25 nucleotides to full length. The aptamer may comprise, consist essentially of, or consist of any span of contiguous nucleotides from SEQ ID NO: 38, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 contiguous nucleotides in the nucleic acid sequence of SEQ ID NO: 39. The aptamer may comprise, consists essentially of, or consist of any span of contiguous nucleotides from SEQ ID NO: 39, where the span has a length chosen in one nucleotide increments from 25 nucleotides to full length. The aptamer may comprise, consist essentially of, or consist of any span of contiguous nucleotides from SEQ ID NO: 39, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, or 35 contiguous nucleotides in the nucleic acid sequence of SEQ ID NO: 43. The aptamer may comprise, consists essentially of, or consist of any span of contiguous nucleotides from SEQ ID NO: 43, where the span has a length chosen in one nucleotide increments from 25 nucleotides to full length. The aptamer may comprise, consist essentially of, or consist of any span of contiguous nucleotides from SEQ ID NO: 43, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 contiguous nucleotides in the nucleic acid sequence of SEQ ID NO: 44. The aptamer may comprise, consists essentially of, or consist of any span of contiguous nucleotides from SEQ ID NO: 44, where the span has a length chosen in one nucleotide increments from 25 nucleotides to full length. The aptamer may comprise, consist essentially of, or consist of any span of contiguous nucleotides from SEQ ID NO: 44, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 contiguous nucleotides in the nucleic acid sequence of SEQ ID NO: 45. The aptamer may comprise, consists essentially of, or consist of any span of contiguous nucleotides from SEQ ID NO: 45, where the span has a length chosen in one nucleotide increments from 25 nucleotides to full length. The aptamer may comprise, consist essentially of, or consist of any span of contiguous nucleotides from SEQ ID NO: 45, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, or 77 contiguous nucleotides in the nucleic acid sequence of SEQ ID NO: 46. The aptamer may comprise, consists essentially of, or consist of any span of contiguous nucleotides from SEQ ID NO: 46, where the span has a length chosen in one nucleotide increments from 25 nucleotides to full length. The aptamer may comprise, consist essentially of, or consist of any span of contiguous nucleotides from SEQ ID NO: 46, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 contiguous nucleotides in the nucleic acid sequence of SEQ ID NO: 47. The aptamer may comprise, consists essentially of, or consist of any span of contiguous nucleotides from SEQ ID NO: 47, where the span has a length chosen in one nucleotide increments from 25 nucleotides to full length. The aptamer may comprise, consist essentially of, or consist of any span of contiguous nucleotides from SEQ ID NO: 47, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 contiguous nucleotides in the nucleic acid sequence of SEQ ID NO: 48. The aptamer may comprise, consists essentially of, or consist of any span of contiguous nucleotides from SEQ ID NO: 48, where the span has a length chosen in one nucleotide increments from 25 nucleotides to full length. The aptamer may comprise, consist essentially of, or consist of any span of contiguous nucleotides from SEQ ID NO: 48, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 contiguous nucleotides in the nucleic acid sequence of SEQ ID NO: 49. The aptamer may comprise, consists essentially of, or consist of any span of contiguous nucleotides from SEQ ID NO: 49, where the span has a length chosen in one nucleotide increments from 25 nucleotides to full length. The aptamer may comprise, consist essentially of, or consist of any span of contiguous nucleotides from SEQ ID NO: 49, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76 contiguous nucleotides in the nucleic acid sequence of SEQ ID NO: 50. The aptamer may comprise, consists essentially of, or consist of any span of contiguous nucleotides from SEQ ID NO: 50, where the span has a length chosen in one nucleotide increments from 25 nucleotides to full length. The aptamer may comprise, consist essentially of, or consist of any span of contiguous nucleotides from SEQ ID NO: 50, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, or 32 contiguous nucleotides in the nucleic acid sequence of SEQ ID NO: 51. The aptamer may comprise, consists essentially of, or consist of any span of contiguous nucleotides from SEQ ID NO: 51, where the span has a length chosen in one nucleotide increments from 25 nucleotides to full length. The aptamer may comprise, consist essentially of, or consist of any span of contiguous nucleotides from SEQ ID NO: 51, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, or 37 contiguous nucleotides in the nucleic acid sequence of SEQ ID NO: 52. The aptamer may comprise, consists essentially of, or consist of any span of contiguous nucleotides from SEQ ID NO: 52, where the span has a length chosen in one nucleotide increments from 25 nucleotides to full length. The aptamer may comprise, consist essentially of, or consist of any span of contiguous nucleotides from SEQ ID NO: 52, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, or 84 contiguous nucleotides in the nucleic acid sequence of SEQ ID NO: 53. The aptamer may comprise, consists essentially of, or consist of any span of contiguous nucleotides from SEQ ID NO: 53, where the span has a length chosen in one nucleotide increments from 25 nucleotides to full length. The aptamer may comprise, consist essentially of, or consist of any span of contiguous nucleotides from SEQ ID NO: 53, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81 contiguous nucleotides in the nucleic acid sequence of SEQ ID NO: 54. The aptamer may comprise, consists essentially of, or consist of any span of contiguous nucleotides from SEQ ID NO: 54, where the span has a length chosen in one nucleotide increments from 25 nucleotides to full length. The aptamer may comprise, consist essentially of, or consist of any span of contiguous nucleotides from SEQ ID NO: 54, where the span has a length chosen in one nucleotide increments from 10 nucleotides to full length.

In some embodiments, the aptamer comprises, consists essentially of, or consists of 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, or 53 contiguous nucleotides in the nucleic acid sequence of SEQ ID NO: 55. The aptamer may comprise, consists essentially of, or consist of any span of contiguous nucleotides from SEQ ID NO: 55, where the span has a length chosen in one nucleotide increments from 25 nucleotides to full length. The aptamer may comprise, consist essentially of, or consist of any span of contiguous nucleotides from SEQ ID NO: 55, where the span has a length chosen in one nucleotide increments from 10 nucleotides to full length.

These sequences relate to aptamer fragments with equivalent, suitable, or improved binding to a target as described herein as compared to full-length aptamer.

In embodiments, aptamers comprise, consist essentially of, or consist of a nucleic acid sequence comprising at least about 10, 15, 20, 25, 30, 35, 40, 45, 50, 51, 52, 53, 54, 55, 60, 65, 70, 75, 80 or more consecutive nucleotides of a sequence having at least 85%, at least 90%, at least 95%, at least 96%, at least 97%, at least 98%, at least 99% or more identity with any of SEQ ID NOs: 1 to 11, 13 to 14, or 53 to 54. In embodiments, aptamers comprise, consist essentially of, or consist of a nucleic acid sequence comprising at least about 10, 15, 20, 25, 30, 35, 40, 45, 50, 51, 52, 53, 54, 55, 60, 65, 70, 75 or more consecutive nucleotides of a sequence having at least 85%, at least 90%, at least 95%, at least 96%, at least 97%, at least 98%, at least 99% or more identity with any of SEQ ID Nos: 12, 46 or 50. In embodiments, aptamers comprise, consist essentially of, or consist of a nucleic acid sequence comprising at least about 10, 15, 20, 25, 30, 35, 40, 45, 50, 51, 52, 53, 54, 55, 60, or more consecutive nucleotides of a sequence having at least 85%, at least 90%, at least 95%, at least 96%, at least 97%, at least 98%, at least 99% or more identity with any of SEQ ID Nos: 36, 48 or 49. In embodiments, aptamers comprise, consist essentially of, or consist of a nucleic acid sequence comprising at least about 10, 15, 20, 25, 30, 35, 40, 45, 50, 51, 52, 53, 54, 55, 60, or more consecutive nucleotides of a sequence having at least 85%, at least 90%, at least 95%, at least 96%, at least 97%, at least 98%, at least 99% or more identity with any of SEQ ID Nos: 29, 31, 32, 38, 39, 47 or 55. In embodiments, aptamers comprise, consist essentially of, or consist of a nucleic acid sequence comprising at least about 10, 15, 20, 25, 30, 35, 40, 45, 50, or more consecutive nucleotides of a sequence having at least 85%, at least 90%, at least 95%, at least 96%, at least 97%, at least 98%, at least 99% or more identity with any of SEQ ID Nos: 27, 29, 31, 32, 38, 39, 47, or 55. In embodiments, aptamers comprise, consist essentially of, or consist of a nucleic acid sequence comprising at least about 10, 15, 20, 25, 30, 35, 40 or more consecutive nucleotides of a sequence having at least 85%, at least 90%, at least 95%, at least 96%, at least 97%, at least 98%, at least 99% or more identity with any of SEQ ID Nos: 25, 30, 37, 44, or 45. In embodiments, aptamers comprise, consist essentially of, or consist of a nucleic acid sequence comprising at least about 10, 15, 20, 25, 30 or more consecutive nucleotides of a sequence having at least 85%, at least 90%, at least 95%, at least 96%, at least 97%, at least 98%, at least 99% or more identity with any of SEQ ID Nos: 23, 24, 26, 34, 43, 51, or 52. In embodiments, aptamers comprise, consist essentially of, or consist of a nucleic acid sequence comprising at least about 10, 15, 20 or more consecutive nucleotides of a sequence having at least 85%, at least 90%, at least 95%, at least 96%, at least 97%, at least 98%, at least 99% or more identity with any of SEQ ID Nos: 28 or 35. In this context the term “about” typically means the referenced nucleotide sequence length plus or minus 10% of that referenced length.

Aspects of the disclosure relate to a composition comprising two or more aptamers or combination comprising two or more aptamers. Embodiments relate to a composition or combination comprising two or more aptamers, wherein each of the two or more aptamers are independently selected from an aptamer which comprises or consists essentially of a nucleic acid sequence selected from the group consisting of: SEQ. ID. No: 1; SEQ. ID. No: 2; SEQ. ID. No: 3; SEQ. ID. No: 4; SEQ. ID. No: 5; SEQ. ID. No: 6; SEQ. ID. No: 7; SEQ. ID. No: 8; SEQ. ID. No: 9; SEQ. ID. No: 10; SEQ. ID. No: 11; SEQ. ID. No: 12; SEQ. ID. No: 13; SEQ. ID. No: 14; SEQ. ID. No: 23; SEQ. ID. No: 24; SEQ. ID. No: 25; SEQ. ID. No: 26; SEQ. ID. No: 27; SEQ. ID. No: 28; SEQ. ID. No: 29; SEQ. ID. No: 30; SEQ. ID. No: 31; SEQ. ID. No: 32; SEQ. ID. No: 33; SEQ. ID. No: 34; SEQ. ID. No: 35; SEQ. ID. No: 36; SEQ. ID. No: 37; SEQ. ID. No: 38; SEQ. ID. No: 39; SEQ. ID. No: 42; SEQ. ID. No: 43; SEQ. ID. No: 44; SEQ. ID. No: 45; SEQ. ID. No: 46; SEQ. ID. No: 47; SEQ. ID. No: 48; SEQ. ID. No: 49; SEQ. ID. No: 50; SEQ. ID. No: 51; SEQ. ID. No: 52; SEQ. ID. No: 53; SEQ. ID. No: 54; and SEQ. ID. No: 55; or an aptamer comprising or consisting essentially of a nucleic acid sequence which has at least 90%, e.g. 95%, 96%, 97%, 98%, 99% sequence identity with any of SEQ. ID. No. 1 to 14, 23 to 39 and 42 to 55.

Embodiments relate to a composition or combination comprising two or more aptamers wherein one of the two or more aptamers comprises or consists essentially of a nucleic acid sequence as set forth in SEQ. ID. No. 55 and the composition or combination further comprises one or more aptamers which comprise or consist essentially of a nucleic acid sequence as set forth in SEQ. ID. No. 1 to 14, 23 to 39 and 43 to 54.

Embodiments relate to a composition or combination comprising three or more aptamers, wherein each aptamer is independently selected from an aptamer which comprises or consists essentially of a nucleic acid sequence as set forth in SEQ. ID. No. 1 to 14 and 23 to 39 and 43 to 55.

Embodiments relate to a composition or combination which comprises or consists essentially of a nucleic acid sequence as set forth in SEQ. ID. No. 55 and which further comprises two or more aptamers which are independently selected from an aptamer which comprises or consists essentially of a nucleic acid sequence as set forth in SEQ. ID. No. 1 to 14, 23 to 39 and 43 to 54.

Embodiments relate to a composition or combination of three or more aptamers, wherein each aptamer comprises or consists essentially of a nucleic acid sequence as set forth in 1 to 14, 23 to 39 and 43 to 55 or an aptamer comprising or consisting essentially of a nucleic acid sequence which has at least 90%, e.g. 95%, 96%, 97%, 98%, 99% sequence identity with any of SEQ. ID. No. 1 to 14, 23 to 39 and 43 to 55.

Embodiments relate to a composition or combination comprising two or more aptamers wherein one of the two or more aptamers comprises or consist essentially of a nucleic acid sequence as set forth in SEQ. ID. No. 1 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 2 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 3 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 4 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 5 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 6 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 7 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 8 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 9 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 10 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 11 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 12 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 13 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 14 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 23 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 24 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 25 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 26 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 27 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 28 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 29 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 30 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 31 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 32 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 33 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 34 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 35 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 36 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 37 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 38 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 39 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 43 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 44 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 45 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 46 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 47 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 48 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 49 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 50 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 51 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 52 and optionally wherein a second of the two or more aptamers comprises or consist essentially of a nucleic acid sequence as set forth in SEQ. ID. No. 55. Embodiments relate to a composition or combination comprising two or more aptamers wherein one of the two or more aptamers comprises or consist essentially of a nucleic acid sequence as set forth in SEQ. ID. No. 53 and optionally wherein a second of the two or more aptamers comprises or consist essentially of a nucleic acid sequence as set forth in SEQ. ID. No. 55.

Embodiments relate to a composition or combination comprising two or more aptamers wherein one of the two or more aptamers comprises or consist essentially of a nucleic acid sequence as set forth in SEQ. ID. No. 54 and optionally wherein a second of the two or more aptamers comprises or consist essentially of a nucleic acid sequence as set forth in SEQ. ID. No. 55.

Embodiments relate to a composition or combination which comprise the following aptamers: CotE H2.1.2 (SEQ ID NO: 30); CotE D2.1 (SEQ ID NO: 31); CotA C1.1 (SEQ ID NO: 33); and CotEC Chitinase (SEQ ID NO: 36). The aptamers may comprise natural or non-natural nucleotides and/or base derivatives (or combinations thereof). In embodiments, the aptamers comprise one or more modifications such that they comprise a chemical structure other than deoxyribose, ribose, phosphate, adenine (A), guanine (G), cytosine (C), thymine (T), or uracil (U). The aptamers may be modified at the nucleobase, at the sugar or at the phosphate backbone.

In embodiments, the aptamers comprise one or more modified nucleotides. Exemplary modifications include for example nucleotides comprising an alkylation, arylation or acetylation, alkoxylation, halogenation, amino group, or another functional group. Examples of modified nucleotides include, but are not limited to, 2′-fluoro ribonucleotides, 2′-NH 2-, 2′-OCH 3- and 2′-O-methoxyethyl ribonucleotides, which are used for RNA aptamers.

The aptamers may be wholly or partly phosphorothioate or DNA, phosphorodithioate or DNA, phosphoroselenoate or DNA, phosphorodiselenoate or DNA, locked nucleic acid (LNA), peptide nucleic acid (PNA), N3′-P5′phosphoramidate RNA/DNA, cyclohexene nucleic acid (CeNA), tricyclo DNA (tcDNA) or spiegelmer, or the phosphoramidate morpholine (PMO) components or any other modification known to those skilled in the art (see also Chan et al., Clinical and Experimental Pharmacology and Physiology (2006) 33, 533-540).

Some of the modifications may allow the aptamers to be stabilized against nucleic acid-cleaving enzymes. In the stabilization of the aptamers, a distinction can generally be made between the subsequent modification of the aptamers and the selection with already modified RNA/DNA. The stabilization may not affect the affinity of the modified RNA/DNA aptamers but may prevent the rapid decomposition of the aptamers in an organism, biological solutions, or solutions, by RNases/DNases. An aptamer is referred to as stabilized if the half-life of the aptamer in the sample (e.g. biological medium, organism, solution) is greater than one minute, greater than one hour, or greater than one day. The aptamers may be modified with reporter molecules, which may enable detection of the labelled aptamers. Reporter molecules may also contribute to increased stability of the aptamers.

Aptamers form a three-dimensional structure that depends on their nucleic acid sequence. The three-dimensional structure of an aptamer may arise due to Watson and Crick intramolecular base pairing, Hoogsteen base pairing (quadruplex), wobble-pair formation, or other non-canonical base interactions. This structure enables aptamers, analogous to antigen-antibody binding, to bind target structures accurately. A nucleic acid sequence of an aptamer may, under defined conditions, have a three-dimensional structure that is specific to a defined target structure.

Embodiments comprise competitive aptamers that compete for binding to a target as defined herein with aptamers as described herein. Embodiments comprise competitive aptamers that compete for binding to a target as defined herein with the aptamers set forth in any one of SEQ ID NOs: 1 to 14, 23 to 39, or 43 to 55, or with aptamers having a nucleic acid sequence having at least 85%, at least 90%, at least 95%, at least 96%, at least 97%, at least 98%, at least 99% sequence identity to the nucleotide sequence of any one of SEQ ID NOs: 1 to 14, 23 to 39, or 43 to 55. Embodiments comprise competitive one or more aptamers that compete for binding to a target as defined herein with one or more of the aptamers described above. In embodiments, competition assays may be used identify a competitive aptamer that competes for binding to a target as defined herein. In an exemplary, non-limiting, competition assay, an immobilized target as defined herein is incubated in a solution comprising a first labelled aptamer that binds to a target as defined herein and a second unlabelled aptamer that is being tested for its ability to compete with the first aptamer for binding to a target as defined herein. As a control, an immobilized target as defined herein may be incubated in a solution comprising the first labelled aptamer but not the second unlabelled aptamer. After incubation under conditions permissive for binding of the first aptamer to a target as defined herein excess unbound aptamer may be removed, and the amount of label associated with immobilized target as defined herein measured. If the amount of label associated with immobilized target as defined herein is substantially reduced in the test sample relative to the control sample, then that indicates that the second aptamer is competing with the first aptamer for binding to a target as defined herein.

### Methods

Embodiments of the present disclosure relate to a method for killing or inactivating C. difficile spores, comprising contacting the spores with a sporicidal solution and an aptamer.

In embodiments, the method is for enhancing the sporicidal effect of the sporicidal solution and comprises contacting C. difficile spores with an aptamer as described herein, prior to, simultaneously with or subsequent to the sporicidal solution. Embodiments of the present disclosure relate to a method which comprises a first step of determining whether C. difficile is located at a location e.g. on a surface.

In the context of the present disclosure the term “killing or inactivating spores” is intended to mean that at least 85%, e.g. 86, 87, 88, 89, 90, 91, 92, 93, 94, 95, 96, 97, 98 or at least 99% of the spores are not capable of transforming (germinating) into vegetative cells.

In some embodiments, 99.9% of the spores are not capable of transforming into vegetative cells. In an embodiment, 99.99% or 99.999% of the spores are not capable of transforming into vegetative cells. In some embodiments, at least 85% of the spores are not capable of transforming into vegetative cells.

The C. difficile colonization may be comprised of a single strain or may be a mixture of strains. For example, the C. difficile may be a strain of a Clade selected from Clade 1, 2, 3, 4 and 5. In certain embodiments, the strain is selected from SH7 (Clade 1), SH8 (Clade 2), R20291 (Clade 2), SH9 (Clade 3), SH10 (Clade 4), ATCC 43598 (Clade 4) and SH11 (Clade 5). In certain embodiments, the strain is selected from R20291, ATCC 43598 and SH11.

The spores may be contacted at a temperature between 0 °C and 90 °C, e.g. between 5 °C and 80 °C, e.g. between 10 °C and 70 °C, between 15 °C and 60 °C, between 18 °C and 50 °C, or between 20 °C and 40 °C, e.g. 20 °C, 21 °C, 22 °C, 23 °C, 24 °C, 25 °C, 26 °C, 27 °C, 28 °C, 29 °C, 30 °C, 31 °C, 32 °C, 33 °C, 34 °C, 35 °C, 36 °C, 37 °C, 38 °C, 39 °C or 40 °C.

In embodiments, the method is performed at a temperature of between about 5 °C to about 90 °C e.g. 5 °C, 10 °C, 15 °C, 20 °C, 25 °C, 30 °C, 35 °C, 40 °C, 45 °C, 50 °C, 55 °C, 60 °C, 65 °C, 70 °C, 75 °C, 80 °C or 90 °C. In embodiments, the method may be performed at a temperature in the range of about 5 °C to about 90 °C. In embodiments, the method may be performed at a temperature in the range of about 5 °C to 10 °C, 10 °C to 15 °C, 15 °C to 20 °C, 20 °C to 25 °C, 25 °C to 30 °C, 30 °C to 35 °C, 35 °C to 40 °C, 40 to 45 °C, 45 °C to 50 °C, 50 °C to 55 °C, 55 °C to 60 °C, 60 °C to 65 °C, 65 °C to 70 °C, 70 °C to 75 °C, 75 °C to 80 °C, 80 °C to 85 °C, 85 °C to 90 °C, or any interval between any of these temperature ranges. In an embodiment, the heating temperatures are selected from 1 °C increments selected from the range of 5 °C to 90 °C.

Certain embodiments of the disclosure provide methods and kits which are suitable for killing or inactivating spores in a variety of environments. Certain embodiments of the disclosure provide methods and kits which are suitable for enhancing the sporicidal effect against C. difficile spores of a sporicidal solution in a variety of environments. The methods and products of certain embodiments may be used in any environment to reduce spore contamination, such as the health-care industry (e.g. animal hospitals, human hospitals, animal clinics, human clinics, nursing homes, day-care facilities for children or senior citizens, etc.), the food industry (e.g. restaurants, food-processing plants, food-storage plants, grocery stores, etc.), the hospitality industry (e.g. hotels, motels, resorts, cruise ships, etc.), the education industry (e.g. schools and universities), etc.

The composition of the disclosure may desirably be used in any environment to reduce spore contamination, such as general-premise surfaces (e.g. floors, walls, ceilings, exterior of furniture, etc.), specific-equipment surfaces (e.g. hard surfaces, manufacturing equipment, processing equipment, etc.), textiles (e.g. cottons, wools, silks, synthetic fabrics such as polyesters, polyolefins, and acrylics, fiber blends such as cotton polyester, etc.), wood and cellulose-based systems (e.g. paper), soil, animal carcasses (e.g. hide, meat, hair, feathers, etc.), foodstuffs (e.g. fruits, vegetables, nuts, meats, etc.), and water.

In one embodiment, the method of the disclosure is directed to sporicidal treatment of textiles. Non-limiting examples of textiles that can be treated with the composition of the disclosure include, but are not limited to, personal items (e.g. shirts, pants, stockings, undergarments, etc.), institutional items (e.g. towels, lab coats, gowns, aprons, etc.), hospitality items (e.g. towels, napkins, tablecloths, etc.).

The surface may be an inanimate surface. The surface may be for example a surface located in a healthcare setting e.g. a hospital, a pharmacy, a doctor's surgery and/or care home facility. The aptamers may be for use in killing and/or inactivating (and optionally first detecting) C. difficile spores located on a surface of an object in an environment selected from the group consisting of a school, a prison, a hostel, a dormitory, a train, a bus and an airplane.

The surface may be located on an object in a location such as a hospital or other healthcare setting. For example, the object may be selected from the group consisting of an operating table, a hospital bed, a surgical instrument, a table, operating scrubs, a refuse container, eating utensils, a chair, a door handle and a doorknob. Alternatively, or in addition, the surface may be located in a location such as walls, ceilings and/or floors in e.g. hospital wards, operating theatres, care home rooms and the like.

In embodiments, the surface may be a surface of an object in a community setting for example a shop, a bar and/or a restaurant. In non-limiting examples, the surface may be located on a wall, a floor, an item of furniture, cutlery, packaging, drinking vessels and the like.

In embodiments, the surface is located in a household environment.

In embodiments, the surface is located in a food production facility.

In certain embodiments, the surface is composed of stainless steel. In certain embodiments, the surface is composed of cardboard. In an embodiment, the surface is composed of plastic.

In certain embodiments, the method comprises contacting a composition comprising an aptamer according to the present disclosure with a surface directly. That is to say, in certain embodiments, the method comprising applying a composition comprising the aptamer to the surface. In embodiments, the method comprises contacting a composition comprising an aptamer according to the present disclosure with a surface indirectly.

In embodiments in which the aptamer is contacted with the surface directly, the method may comprise providing the aptamer in a composition which is capable of being dispersed across the surface. In certain embodiments, immediate contact with the surface dispersed the composition uniformly for maximum surface area coverage and wettability facilitates the accessibility of aptamer to target molecules on the C. difficile spore.

Most environmental surface areas are neutral (uncharged) or have negative electrostatic energy. In certain embodiments, the aptamer is applied to a target surface area using electrostatic force of attraction. It is considered that electrostatically applied liquids have a wrapping effect, so that complex objects and areas hidden from the line of sight get coated with the liquid.

Based on Coulomb's law, an electrostatic application system applies aptamer/buffer solutions more evenly to all surfaces. Coulomb's law states that the magnitude of the electrostatic force of interaction between two-point charges is directly proportional to the scalar multiplication of the square of the distance between them. The force is along the straight line joining them. Charged spray droplets are attracted to surfaces and are considered to have an enveloping effect around the object to insure all sides are covered.

Using Coulomb's law, these systems place a positive or negative charge on the chemical solution as it leaves the spray nozzle. Because most surface areas are neutral or negative, a positively charged electrostatic spray application system optimizes adhesion and attraction. The dispersed droplets may spread out more evenly and seek out the negative (−) or neutrally charged surface. Thus in certain embodiments, the composition comprising the aptamers as described herein is more targeted and provides more consistent coverage with less waste.

In embodiments, the method comprises applying a composition comprising an aptamer to a surface by a spray gun modified with an electrode. The electrode charges liquid droplets comprising the aptamer which are then guided to the surface, which is typically oppositely charged to the aptamer.

In embodiments, the method comprises producing a composition comprising the aptamer. In embodiments, the aptamers of the presently claimed disclosure are provided in a dried form and are dissolved completely to a desired stock concentration with a buffer solution or dH2O. This may be achieved by, for example, shaking the composition for a predetermined period of time e.g. 20 minutes, 25 minutes, 30 minutes or more e.g. 35 minutes, 40 minutes, 45 minutes or greater. The composition may comprise an organic solvent e.g. DMSO, ethanol and/or methanol. Additionally, the composition may comprise a salt such as for example a sodium ion.

In embodiments, the method comprises dissolving the aptamer in a buffer solution. The buffer solution may be selected from, for example PBS, HEPES, Tris etc. Typically, aptamers are stable at neutral pH range (7.0-8.0). A heating and a cooling step may be performed for the proper folding of aptamer structure in a buffer solution (for example heating at 95 °C for 5 min followed by slow cooling to room temperature). In certain embodiments, the method comprises providing a 2+ ion such as magnesium in the buffer solution. Divalent ions such as magnesium may be advantageous in certain embodiments to maintain a proper structure of the aptamer.

Nucleic acid aptamer in the binding buffer condition (pH 7.4) is negatively charged and through electrostatic interactions the aptamer could favor binding to positively charged areas. To avoid this interaction with aptamer/buffer and container the use of anti-static treated materials (e.g., plastic and glass vessels) are important. Typically, the sprayer will be made of plastics.

In embodiments, the method comprises applying a composition comprising the aptamer to the surface by spraying the surface.

In embodiments, the aptamers may be freeze-dried prior to being sprayed onto the surface. Thus, in certain embodiments the method comprises a step of contacting the surface with a freeze-dried composition comprising the aptamer as described herein.

During the spray freezing step, the aptamer dissolved or suspended in liquid is atomized into fine droplets which are frozen instantaneously by a cryogenic fluid, usually liquid nitrogen. Subsequently, the frozen particles are subjected to freeze drying, in which the solvents are sublimed at low temperature and pressure, leading to the formation of dried porous particles. Porous particles with large physical size and low density exhibit small aerodynamic size, which can promote high flowability. The small contact surface area to volume ratio leads to low cohesion force between particles, thereby facilitates dispersibility in air. In addition, porous particles have high specific surface area, thereby enhancing dissolution rate.

In embodiments, the method comprises dissolving freeze-dried aptamers in a solution prior to contacting the surface with a liquid solution.

In embodiments, the method of killing and/or inactivating C. difficile at a location, e.g. a surface, may comprise applying one or more of the aptamers of the disclosure to a location suspected of comprising C. difficile spores. Following a predetermined period of time sufficient to permit the aptamer binding to C. difficile spores, the surface may be washed one or more times to remove any unbound aptamer. In embodiments, e.g. when a FRET pair is used or beacon as described herein, the washing step is not required.

In certain embodiments, for use on environmental surfaces such as stainless steel, polystyrene and other surfaces the aptamers are designed to attach to the target, i.e. a C. difficile spores, and fluoresce. The aptamers will not fluoresce by attaching to the inorganic surface alone.

### Sporicidal Agent

Embodiments of the disclosure comprise the use of sporicidal agents e.g. sporicidal solutions. Bacterial spores are considered to be highly resistant to chemical and physical agents. Embodiments of the present disclosure aim to increase the sensitivity of the spore to sporicidal agents.

As used herein, the term “sporicidal agent” refers to an agent which is capable of killing C. difficile spores. In certain embodiments, the methods described herein may replace a sporicidal agent with a sporostatic agent, i.e. an agent which is inhibitory to spore germination or outgrowth or both. Certain agents may be sporostatic at certain concentrations and/or temperatures and sporicidal at higher concentrations and/or temperatures.

Exemplary sporicidal agents include but are not limited to glutaraldehyde, sodium hypochlorite, iodine, iodophors, hydrogen peroxide and peracetic acid.

The sporicidal solution for use in embodiments of the present disclosure may be an alkylating agent, and oxidizing agent and/or a chlorine-releasing agent.

In embodiments, the sporicidal agent is a solution. In embodiments, the sporicidal agent comprises an agent which, on addition of water, generates peracetic acid. In embodiments, the sporicidal agent is a solution comprising peracetic acid. The solution may be formed by wetting dry wipes (such as wipes marketed under the Clinell trade name) and squeezing solution from the wetted wipe.

In certain embodiments, the sporicidal agent is formed from a wipe comprising Sodium Percarbonate at a concentration of ≤50 (% wt) (Cas No. 239-707-6 15630-89-4) and Citric Acid (concentration ≤20 (% wt) (Cas No. 77-92-9 201-069-1). The wipe may also comprise tetra acetyl ethylene diamine (concentration ≤25% wt.) In embodiments, the sporicidal agent is a solution comprising peracetic acid.

In embodiments, the sporicidal agent comprises an agent which is capable of generating peracetic acid. In embodiments, the sporicidal agent comprises sodium percarbonate.

In embodiments, the sporicidal agent may be a wipe sold under Mikrozid® brand name.

In embodiments, the sporicidal agent comprises peracetic acid (e.g. at a concentration of about 0.05-0.10% in a 100 g agent solution) and hydrogen peroxide. The sporicidal agent may further comprise acetic acid. In certain embodiments, the agent comprises around 0.06% peracetic acid.

In embodiments, the sporicidal solution comprises sodium hypochlorite. The solution may comprise sodium hypochlorite in a concentration of between about 0.3 to about 0.7% v.v. The solution may be pre-diluted to form the sporicidal solution for use in the methods of embodiments of the disclosure.

In embodiments, the sporicidal solution may comprise hydrogen peroxide e.g. about 1.5% (Aseptix Sterimax Sporicide wipes). In embodiments, the solution comprises a silver-stabilised hydrogen peroxide e.g. a solution sold under the trademark EndoSan®.

In embodiments, the sporicidal solution comprises chlorhexidine digluconate. Aptly, the sporicidal solution is a solution available under the brand name Chemgene™.

In embodiments, the sporicidal solution comprises glucoprotamin 1.5% (Incidin plus wipes); a mixture of ethanol, propane and N-alkyl amino propyl glycine (Bacillol 30 tissues); and finally, a mixture of didecyldimonium chloride, benzalkonium chloride, polyaminopropyl, biguanide and dimenthicone as active ingredients (Formula 429 spray).

### Detectable Labels

In embodiments, the aptamers of the disclosure are used to detect and/or quantify the amount of C. difficile at a location. In embodiments, the methods comprise a step of determining the presence, absence and/or concentration of C. difficile at a surface prior to contacting the location with a sporicidal agent (i.e. kill the spore) and/or sporistatic agent (e.g. agent that inhibits spore germination or outgrowth). The step of determining the presence, absence and/or concentration comprises contacting the location with one or more aptamers as defined herein for a period of time to enable an aptamer-spore complex to form.

In embodiments, the aptamer comprises a detection molecule. Any label capable of facilitating detection and/or quantification of the aptamers may be used herein.

In embodiments, the detectable label is a fluorescent moiety, e.g. a fluorescent/quencher compound. Fluorescent/quencher compounds are known in the art. See, for example, Mary Katherine Johansson, Methods in Molecular Biol. 335: Fluorescent Energy Transfer Nucleic Acid Probes: Designs and Protocols, 2006, Didenko, ed., Humana Press, Totowa, NJ, and Marras et al., 2002, Nucl. Acids Res. 30, e122 (incorporated by reference herein).

In embodiments, the detectable label is FAM. In certain embodiments, the FAM-label is situated at the first and/or second primer region of the aptamer. The person skilled in the art would understand that the label could be located at any suitable position within the aptamer.

Moieties that result in an increase in detectable signal when in proximity of each other may also be used herein, for example, as a result of fluorescence resonance energy transfer (“FRET”); suitable pairs include but are not limited to fluoroscein and tetramethylrhodamine; rhodamine 6G and malachite green, and FITC and thiosemicarbazole, to name a few.

In embodiments, the detectable label is selected from at least one of the following non-limiting examples: a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

In embodiments, the detectable label is a fluorescent protein such as Green Fluorescent Protein (GFP) or any other fluorescent protein known to those skilled in the art.

In embodiments, the nature of the detection will be dependent on the detectable label used. For example, the label may be detectable by virtue of its color, e.g. gold nanoparticles. A color can be detected quantitatively by an optical reader or camera e.g. a camera with imaging software.

In embodiments, the detectable label is a fluorescent label, e.g. a quantum dot. In such embodiments, the detection means may comprise a fluorescent plate reader, strip reader or similar, which is configured to record fluorescence intensity.

In embodiments in which the detectable label is an enzyme label, the detection means may, for example, be colorimetric, chemiluminescence and/or electrochemical (for example, using an electrochemical detector). Electrochemical sensing is through conjugation of a redox reporter (including but not limited to methylene blue or ferrocene) to one end of the aptamer and a sensor surface to the other end. Typically, a change in aptamer conformation upon target binding changes the distance between the reporter and sensor to provide a readout.

In embodiments, the detectable label may further comprise enzymes such as horseradish peroxidase (HRP), Alkaline phosphatase (APP) or similar, to catalytically turnover a substrate to give an amplified signal.

In embodiments, the disclosure provides a complex (e.g. conjugate) comprising aptamers of the disclosure and a detectable molecule. Typically, the aptamers of the disclosure are covalently or physically conjugated to a detectable molecule.

In embodiments, the detectable molecule is a visual, optical, photonic, electronic, acoustic, opto-acoustic, mass, electrochemical, electro-optical, spectrometric, enzymatic, or otherwise physically, chemically or biochemically detectable label.

In embodiments, the detectable molecule is detected by luminescence, UV/VIS spectroscopy, enzymatically, electrochemically or radioactively. Luminescence refers to the emission of light. For example, photoluminescence, chemiluminescence and bioluminescence are used for detection of the label. In photoluminescence or fluorescence, excitation occurs by absorption of photons. Exemplary fluorophores include, without limitation, bisbenzimidazole, fluorescein, acridine orange, Cy5, Cy3 or propidium iodide, which can be covalently coupled to aptamers, tetramethyl-6-carboxyhodamine (TAMRA), Texas Red (TR), rhodamine, Alexa Fluor dyes (et al. Fluorescent dyes of different wavelengths from different companies).

In certain embodiments, the detectable molecule is one of a FRET pair. In certain embodiments, the aptamer may comprise a FRET pair as detectable molecules. Binding of the aptamer to the C. difficile spore may result in a loss of quenching which is detectable.

In embodiments, the detectable molecule is a colloidal metallic particle, e.g. gold nanoparticle, colloidal non-metallic particle, quantum dot, organic polymer, latex particle, nanofiber (e.g. carbon nanofiber), nanotube (e.g. carbon nanotube), dendrimer, protein or liposome with signal-generating substances. Colloidal particles can be detected colorimetrically.

In embodiments, the detectable molecule is an enzyme. In certain embodiments, the enzyme may convert substrates to coloured products, e.g. peroxidase, luciferase, β-galactosidase or alkaline phosphatase. For example, the colorless substrate X-gal is converted by the activity of β-galactosidase to a blue product whose color is visually detected.

In embodiments, the detection molecule is a radioactive isotope. The detection can also be carried out by means of radioactive isotopes with which the aptamer is labelled, including but not limited to 3H, 14C, 32P, 33P, 35S or 125I, more preferably 32P, 33P or 125I. In the scintillation counting, the radioactive radiation emitted by the radioactively labelled aptamer target complex is measured indirectly. A scintillator substance is excited by the isotope's radioactive emissions. During the transition of the scintillation material, back to the ground state, the excitation energy is released again as flashes of light, which are amplified and counted by a photomultiplier.

In embodiments, the detectable molecule is selected from digoxigenin and biotin. Thus, the aptamers may also be labelled with digoxigenin or biotin, which are bound for example by antibodies or streptavidin, which may in turn carry a label, such as an enzyme conjugate. The prior covalent linkage (conjugation) of an aptamer with an enzyme can be accomplished in several known ways. Detection of aptamer binding may also be achieved through labelling of the aptamer with a radioisotope in an RIA (radioactive immunoassay). In an embodiment, the radioisotope is 125I. Detection of aptamer binding may also be achieved through labelling of the aptamer by fluorescence in a FIA (fluoroimmunoassay) with fluorophores. In an embodiment, the fluorophore is fluorescein or FITC.

In some embodiments, antisense oligonucleotides can be designed to hybridize that are complementary to the 5′ end, the 3′ end, the 5′ end and the 3′ end of or any relevant sequence of the aptamer (e.g. H2.1.2-as SEQ ID NOs: 40-41) In some embodiments, the antisense oligonucleotides comprises a fluorophore.

Embodiments comprise methods for detecting the presence, absence or amount of a target as defined herein at a location e.g. a surface. In the methods, the surface may be interacted (i.e. contacted) with an aptamer as described herein. For example, the surface and aptamers as described herein may be incubated under conditions sufficient for at least a portion of the aptamer to bind to a target as defined herein in the sample.

A person skilled in the art will understand that the conditions required for binding to occur between the aptamers described herein and a target as defined herein. In embodiments the sample e.g. a target surface at a location suspected of including C. difficile spores, and aptamer may be incubated at temperatures between about 4 °C and about 40 °C. In embodiments the sample and aptamer may be incubated at temperatures between about 20 °C and about 37 °C. In embodiments the sample and aptamer may be incubated at 22 °C. The incubation temperature may be selected from the range of 4 °C to less than 20 °C, 20 °C to less than 22 °C, 22 °C to less than 24 °C, 24 °C to less than 26 °C, 26 °C to less than 28 °C, 28 °C to less than 30 °C, 30 °C to less than 32 °C, 32 °C to less than 34 °C, 34 °C to less than 36 °C, 36 °C to less than 37 °C and 37 °C to less than 40 °C. In embodiments, the sample and aptamer may be diluted to different concentrations (e.g. at least about 1%, 5%, 10%, 20%, 25%, 30%, 40%, 50%, 60%, 70% 80% v/v or more) with a buffer (exemplary buffers include but are not limited to PBS). The diluted concentrations may be selected from the range of 1% to less than 5%, 5% to less than 10%, 10% to less than 20%, 20% to less than 30%, 30% to less than 40%, 40% to less than 50%, 50% to less than 60%, 60% to less than 70%, 70% to less than 80%, or 80% to less than 90% v/v. In embodiments, the aptamer concentration before dilution may be from 100 nM to 50 μM. In embodiments, the aptamer concentration before dilution may be selected from the range of 100 nM to 500 nM, 500 nM to 1 μM, 1 μM to 2 μM, 2 μM to 5 μM, 5 μM to 10 μM, 10 μM to 15 μM, 15 μM to 20 μM, 20 μM to 30 μM, 30 μM to 40 μM, 40 μM to 50 μM, 50 μM to 60 μM, 60 μM to 70 μM, 70 μM to 80 μM, 80 μM to 90 μM, 90 μM to 100 μM. In embodiments, the aptamer concentration before dilution may be a concentration selected from the ranges described herein. The selected value may be selected from 0.1 μM increment concentrations in a range herein. In embodiments, the aptamer concentration before dilution may be 2 μM. In embodiments, the aptamer concentration used for binding may be selected from the range of 10 μM to 40 μM, In embodiments, the aptamer concentration used for binding may be selected from the range of 10 μM to 15 μM, 15 μM to 20 μM, 20 μM to 25 μM, 25 μM to 30 μM, 30 μM to 35 μM, 35 μM to 40 μM, or any concentration between any of these concentration ranges selected from 1 μM increments selected from the range of 10 μM to 40 μM. In an embodiment, the aptamer concentration used for binding may be 20 μM. In an embodiment, the aptamer concentration used for binding may be 25 μM. In an embodiment, the aptamer concentration used for binding may be 30 μM. In embodiments, the sample and aptamer may be incubated whilst shaking and/or mixing. In certain embodiments, the sample and aptamer are incubated for at least 1 minute, at least 5 minutes, at least 15 minutes, at least 1 hour or more. The sample and aptamer may be incubated for 1 minute to less than 5 minutes, 5 minutes to less than 15 minutes, 15 minutes to less than one hour, one hour to less than 24 hours, 24 hours to less than 48 hours.

In embodiments, binding of the aptamer and a target as defined leads to formation of an aptamer-target complex. The binding or binding event may be detected, for example, visually, optically, photonically, electronically, acoustically, opto-acoustically, by mass, electrochemically, electro-optically, spectrometrically, enzymatically or otherwise chemically, biochemically or physically as described herein.

The binding of aptamer and the target may be detected using any suitable technique. As discussed above, for example, binding of the aptamer and the target may be detected using a biosensor. In embodiments, binding of the aptamer and the target is detected using SPR, RlfS, BLI, LFD or ELONA as described herein.

In embodiments, the method comprises a step of determining the binding of the aptamer to a C. difficile spore and subsequently a step of determining killing and/or inactivation of C. difficile spores. In embodiments, the method comprises visualising the killing and/or inactivation of C. difficile spores. Such visualization may be in the form of a loss of a detectable signal, e.g. the reduction or loss of a visual, optical, photonic, electronic, acoustic, opto-acoustic, mass, electrochemical, electro-optical, spectrometric or enzymatic signal.

In embodiments, the method comprises the use of a light source, such as a laser; focusable optics, such as a lens; filters or monochromators to effect changes in the spectrum of excitation or fluorescence emission; and a CCD camera or a single photon count detector to measure fluorescence to visualise the binding of aptamer to C. difficile spores and/or the killing and/or inactivation of C. difficile spores.

The method may comprise a set of conditions for illuminating the location using a light source. In an embodiment, the light source may be in the form of a forensic light source. In an embodiment, the light source may be in the form of a Polilight® Flare.

In embodiments, the light source may be capable of switching between different wavelengths, each wavelength being suited to a specific interchangeable filter. The forensic light source may be in the form of a LED, laser, Polilight® or the like. In embodiments, the light source is a handheld light source. In an embodiment, the handheld light source may be a Polilight Flare+2, which is a battery operated, handheld LED light source, available from e.g. Rofin Forensic.

In embodiments, each Polilight Flare “torch” may produce light within a specified wavelength range. For example, in embodiments, the light source may produce light at a wavelength of between about 360 nm-385 nm (UV light). In embodiments, the light source may produce light at a wavelength of between about 405 nm-420 nm. In embodiments, the light source may produce light at a wavelength of between about 435 nm-465 nm. In embodiments, the light source may produce light at a wavelength of between about 485 nm-515 nm. In embodiments, the light source may produce light at a wavelength of between about 510 nm-545 nm. In embodiments, the light source may produce light at a wavelength of between about 530 nm-560 nm. In embodiments, the light source may produce light at a wavelength of between about 585 nm-605 nm. In embodiments, the light source may produce light at a wavelength of between about 615 nm-635 nm. In embodiments, the light source may produce light at a wavelength of between about 400 nm-700 nm. In embodiments, the light source may produce light at a wavelength of between about 835 nm-865 nm. In embodiments, the light source may produce light at a wavelength of between about 935 nm-965 nm.

In embodiments, the light source used may be compatible with a detectable molecule conjugated to the aptamer. In embodiments, the aptamer will be conjugated to a detection molecule. In embodiments, the detection molecule may be a fluorophore which emits in a spectral range which corresponds to the output of the light source. In embodiments, the aptamer may be conjugated to a fluorophore which emits at a wavelength of about 505 nm. In embodiments, the light source produces light having a wavelength of about 505 nm.

In embodiments, the method may comprise the use of a bandpass filter in combination with the light source. The bandpass filter may be configured to transmit light of a certain wavelength band and reject stray light outside the predetermined wavelength band. In embodiments, the light source is configured to produce narrow bands of light having centre wavelengths of 365 nm, 415 nm, 450 nm, 505 nm, 530 nm, 545 nm, 620 nm, and 850 nm. In embodiments, the light source is configured to produce narrow bands of light having a center wavelength of 505 nm, in addition to white light wavelengths. In embodiments, the bandpass filter is a 590 nm bandpass filter.

In embodiments, the method may further comprise visualising the location of the surface with viewing goggles, glasses, or the like. In embodiments, the viewing goggles are of a colour which corresponds to the colour of light produced by the light source and emitted by the detection molecule conjugated to the aptamer. In embodiments, the goggles are orange and thus are suitable for use in combination with a light source which produces light having a wavelength of between about 485 nm-515 nm, e.g. 505 nm, and an aptamer which comprises a detection molecule that emits at a wavelength of approximately 505 nm.

### Compositions

In certain aspects of the disclosure, there is provided a combination of one or more aptamers as described herein and a sporicidal and/or sporistatic agent as defined herein. The combination may be for use in methods of killing and/or inactivating C. difficile e.g. C. difficile spores at a location such as a surface. The combination may also be for use in the enhancement of sporicidal activity of the sporicidal agent. The combination may also be for use in the detection of C. difficile spores prior to killing or inactivation.

The aptamers as defined herein and the sporicidal agent may be provided in a single composition. The composition may further comprise other components such as but not limited to a buffer solution, stabilizing agents, colourants and the like.

Alternatively, the aptamer and the sporicidal agent may be provided in separate compositions. In embodiments, the aptamer and the sporicidal agent may be for sequential application to the location.

The disclosure also provides a kit for killing and/or inactivation of C. difficile, wherein the kit comprises one or more aptamers as described herein. In embodiments, the kit also comprises a sporicidal agent. In embodiments, the sporicidal agent is a solution as described herein. In embodiments, the kit also comprises a detectable molecule as described herein.

In some embodiments, the kit further comprises instructions for use in accordance with any of the methods described herein.

Typically, the kit comprises further components for the reaction intended by the kit or the method to be carried out, for example components for an intended detection of enrichment, separation and/or isolation procedures. Examples are buffer solutions, substrates for a color reaction, dyes or enzymatic substrates. In the kit, the aptamer may be provided in a variety of forms, for example pre-immobilised onto a support (e.g. solid support), freeze-dried or in a liquid medium.

The kit of the disclosure may be used for carrying out any method described herein. It will be appreciated that the parts of the kit may be packaged individually in vials or in combination in containers or multi-container units. Typically, manufacture of the kit follows standard procedures which are known to the person skilled in the art.

### EXAMPLES

In the following, the disclosure will be explained in more detail by means of non-limiting examples of specific embodiments. In the example experiments, standard reagents and buffers free from contamination are used.

### Example 1

Demonstration of sporicidal activity of three sporicidal products at five concentrations in the presence and absence of the CotE_H2 aptamer using Clostridioides difficile (previously Clostridium difficile) SH11 spores.

Demonstration of sporicidal activity of three sporicidal products at five concentrations in the presence and absence of three aptamers using two strains of C. difficile spores.

### Materials and Methods

### Test Microorganisms

The Clostridium difficile spore suspensions used in this study are listed in Table 5.

C. difficile SH11 suspensions were provided by SporeGen® and stored at 4 °C upon arrival. Clostridioides difficile R20291 suspension was provided by Sporegen® and stored at 4 °C upon arrival.

TABLE 5
Details of Clostridium difficile
spore suspensions used in this study.

| | | In-test |
|---|---|---|
| Ribotype | Description | Concentration |
| RT027 (R20291) | Purified spore | 1 × 10⁸ ± 5 × 10⁷ |
| | suspension | |
| RT078 (SH11) | Purified spore | 1 × 10⁸ ± 5 × 10⁷ |
| | suspension | |
| ATCC ® 43598 ™ | Internal strain | 1 × 10⁸ ± 5 × 10⁷ |
| | spore suspension | |

### Test Agents

Test agents used are listed in Table 6 and 7. Aptamers and buffers were provided by Aptamer Group Limited.

The sporicidal agents can be obtained as follows: Chemgene™ (a high level laboratory surface disinfectant that combines active ingredient molecules with micelle cleaning technology, biodegradable, non-corrosive-see www.starlabgroup.com/GB-encgloves-safety/laboratory-disinfectantrWebPSub-159946/chemgene-hld4 h-rtu-spray-blue-eucalyptus-1:20-750ml_SLXTM3 02-C)
Endosan® (hard surface silver stabilised hydrogen peroxide see www.clinipathequipment.com/product/endosan-3-trigger-spray
Clinell® (Peracetic acid generating wipes comprising sodium percabonate (≤50% wt, citric acid ≤20 wt % and tetra acetyl ethylene diamine ≤25 wt %—see gamahealthcare.com/products/sporicidal-wipes)

TABLE 6

| | | Concentration | In-test |
|---|---|---|---|
| | | provided | concentration |
| Test Agent | Buffer | (μM) | (μM) |
| CotE_H2 aptamer | TbKst | 100 | 20 |
| CDec_D1 aptamer | TbKat | 100 | 20 |
| CDem_D2 aptamer | TbKat | 100 | 20 |

TABLE 7
Liquid sporicidal test agents and control solutions used throughout the study.
PBS = phosphate buffered saline, RCM = reinforced clostridial medium.

| | | | In-test |
|---|---|---|---|
| | | | concentrations |
| Test Agent | Product Type | Lot Number | (%) |
| Clinell ® sporicidal | Liquid sporicidal | SA613818 | 50, 25, 12.5, 6.25, |
| solution | | | 3.125, 1.5625 |
| EndoSan ® | Liquid sporicidal | 170117HS0300 | 50, 25, 12.5, 6.25, |
| | | | 3.125, 1.5625 |
| Chemgene ™ | Liquid sporicidal | 190592 | 50, 25, 12.5, 6.25, |
| | | | 3.125, 1.5625 |
| Negative control | PBS + 1% RCM | PBS191018/ | N/A |
| | | RCM191018 | |
| Positive control | Bleach-based | 9 161 2696 | N/A |
| | solution | | |

### Equipment and Media

UKAS calibrated pipettes (0.5-1000 μL range), Proline® Plus—Sartorius UK
UKAS calibrated multichannel pipettes (P300-P20)—Gilson UK
Eppendorf 5452 minispin centrifuge—Eppendorf, DE
Calibrated balance, Ohaus NV212—Scientific Laboratories Supplies (SLS) UK
Anaerobic cabinet, Whitley MG500—Don Whitley Scientific Limited, UK
Digital dry bath—SLS, UK
Sterile universal tubes—SLS, UK
96-well plates, SLS, UK

### Media

Nuclease free water—Aptamer Group
TbKat buffer—Aptamer Group
TbKst buffer—Aptamer Group
Brain heart infusion agar supplemented with horse blood and yeast (BHI-YHT).
For components see Table 8
Reinforced clostridial medium (RCM) Acumedia®—SLS, UK
Phosphate buffered saline (PBS)—SLS, UK
GH neutraliser (for components see Table 9)

TABLE 8
Preparation of BHIY-HT

| Chemical Name | Quantity | |
|---|---|---|
| BHIA | 26 | g |
| Yeast Extract | 2.5 | g |
| Sodium Taurocholate Hydrate | 0.5 | g |
| Horse Blood | 35 | mL |
| Distilled Water | 500 | mL |

TABLE 9
Preparation of GH neutraliser

| Compound | Quantity | |
|---|---|---|
| Saponin | 20 | g |
| Sodium thiosulphate | 10 | g |
| Sodium dodecyl sulfate | 5 | g |
| Lecithin | 1 | g |
| L-histidine | 1 | g |
| Tween 80 | 20 | g |
| Distilled water | 1 | L |

### Method

Prior to testing, the CotE_H2 aptamer was diluted in nuclease free water to a 4× stock concentration of 80 μM. The aptamers were folded by heating to 95 °C in a digital dry bath for 5 minutes and then immediately placed in ice and cooled to 2 °C. The CotE_H2 was stored at 2-4 °C while in use. A Clostridium difficile SH11 bacterial spore inoculum was prepared to 1×10⁸±5×10⁷ CFUmL⁻¹ according to internal procedures. The inoculum was confirmed by serial dilution and plating out the resultant suspensions onto brain heart infusion agar supplemented with horse blood and yeast (BHI-YHT). Each 4× stock concentration of CotE-H2 (80 μM) was diluted in TbKst buffer to obtain a 2× in-test concentration (40 μM). Ten microlitres of C. difficile spores were added to 10 μL of 2× in-test concentration of CotE-H2. The spore suspension was pipette mixed and incubated with CotE-H2 at room temperature for one hour. The spores were then washed three times by centrifuging at 13,400 RPM for 10 minutes. In each wash the supernatant was removed, and the pellet was resuspended in 100 μL of “TbKst” buffer. Following the third wash, the spores were resuspended in 100 μL of “TbKst” buffer and vortexed to obtain a homogenous solution.

A total of five 2× in-test concentrations (1:2 dilutions) of each sporicidal test agent were prepared from the stock (100%, 50%, 25%, 12.5%, 6.25%, 3.125%). The Clinell solutions were prepared by adding 100 mL water to the wipes and then squeezing the solution out of them.

Negative and positive control solutions were also prepared. Twenty-five microliter aliquots of each sporicidal test agent concentration or control solution were added to the wells of a 96-well plate. A 25 μL aliquot of the aptamer-spore suspension was added to each test agent or control solution and pipette mixed. The final in-test concentrations of each sporicidal test agent were 50%, 25%, 12.5%, 6.25%, 3.125%, 1.5625%. The plates were then incubated for 15 minutes at 37 °C±2 °C in an anaerobic environment. Following incubation, 50 μL of GH neutraliser was added to each well and pipette mixed. A 10 μL aliquot of neutralised suspension was taken from each well and transferred to an agar plate and incubated for 48 hours at 37 °C±2 °C in an anaerobic environment. Growth was recorded as “+” and no growth as “−”. The minimum sporicidal concentration (MSC) for each concentration was identified as the lowest concentration with no growth for all replicates. If no growth was observed at the lowest concentration it was displayed as “<”. If growth was observed at the highest concentration it was displayed as “>”. The test was then repeated without the aptamers being present. Testing was performed in triplicate.

### Example 2

### The Assessment of the Minimum Sporicidal Concentration of Three Sporicidal Products at Five Concentrations in the Presence and Absence of Three Aptamers Using Two Strains of Clostridium difficile Spores

The methodology outlined in Example 1 was repeated with CDec_D1 and CDem_D2 aptamers for C. difficile SH11 spores and with CotE_H2, CDec_D1 and CDem_D2 aptamers for C. difficile ATCC® 43598™ spores.

### Results

The assessment of the minimum sporicidal concentration of three sporicidal products at five concentrations in the presence and absence of the CotE_H2 aptamer using Clostridium difficile SH11 spores.

When the test agents were assessed against C. difficile SH11 spores without the presence of the CotE_H2 aptamer, the lowest MSC (25%) was reported for Clinell® sporicidal solution (Table 10 and Table 11). A greater than 50% concentration was reported for the MSC of EndoSan® and Chemgene™. When C. difficile SH11 spores were tested in the presence of the CotE_H2 aptamer, a greater than two-fold increase in the MSC was reported for Clinell® sporicidal solution. The other two test products remained unchanged. Particularly, the results indicate that the MSC of the EndoSan® and Chemgene™ products had not been identified. As such, the results indicate that it was not possible to determine whether the presence of aptamers had an effect on the C. difficile spores.

Sporicidal Concentrations of Sporicidal Solutions in the Presence and Absence of Three Aptamers Using Clostridium difficile.

TABLE 10
Growth observed after recovering viable Clostridium difficile
SH11 spores following treatment for 15 minutes with neat and diluted
sporicidal solutions in the absence of CotE_H2 aptamers.
“+” = growth present, “−” = no growth (n = 3).

| | Aptamers absent | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | − | − | − | − | − | − | + | + | + | + | + | + | + | + | + | + | + | + |
| 25 | − | − | − | − | − | − | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

Sporicidal Concentrations of Sporicidal Solutions in the Presence and absence of three aptamers using Clostridium difficile ATCC® 43598™ Spores.

TABLE 11
Growth observed after recovering viable Clostridium difficile
ATCC ® 43598 ™ spores following treatment for
15 minutes with neat and diluted sporicidal solutions in the absence of aptamers.
“+” = growth present, “−“ = no growth (n = 3).

| | Aptamers absent | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | + | + | − | + | + | + | + | + | + | + | + | + | + | + | + | + | + | + |
| 25 | + | + | + | + | + | + | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

TABLE 12
Growth observed after recovering viable Clostridium difficile ATCC ® 43598 ™ spores
following treatment for 15 minutes with neat and diluted sporicidal solutions
in the presence of CotE_H2 aptamers. “+” = growth present, “−” = no growth (n = 3).

| | CotE_H2 present | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | − | − | − | − | − | − | + | + | + | + | + | + | + | + | + | + | + | + |
| 25 | + | + | + | + | + | + | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

TABLE 13
Growth observed after recovering viable Clostridium difficile ATCC ® 43598 ™ spores
following treatment for 15 minutes with neat and diluted sporicidal solutions
in the presence of CDec_D1 aptamers. “+” = growth present, “−” = no growth (n = 3).

| | CDec_D1 present | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | − | − | − | − | − | − | + | + | + | + | + | + | + | + | + | + | + | + |
| 25 | + | + | − | − | + | − | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

TABLE 14
Growth observed after recovering viable Clostridium difficile ATCC ® 43598 ™ spores
following treatment for 15 minutes with neat and diluted sporicidal solutions
in the presence of CDem_D2 aptamers. “+” = growth present, “−” = no growth (n = 3).

| | CDem_D2 present | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | − | − | − | − | − | − | + | + | + | + | + | + | + | + | + | + | + | + |
| 25 | − | − | − | − | − | − | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

Sporicidal Concentrations of Sporicidal Solutions in the Presence of Two Aptamers Using Clostridium difficile SH11 Spores.

TABLE 15
Growth observed after recovering viable Clostridium difficile
SH11 spores following treatment for 15 minutes with neat and diluted
sporicidal solutions in the presence of CDec_D1 aptamers.
“+” = growth present, “−” = no growth (N = 3).

| | CDec_D1 present | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | − | − | − | − | − | − | − | + | + | + | + | + | + | + | + | + | + | + |
| 25 | − | − | − | − | − | − | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

TABLE 16
Growth observed after recovering viable Clostridium difficile
SH11 spores following treatment for 15 minutes with neat and diluted
sporicidal solutions in the presence of CDem_D2 aptamers.
“+” = growth present, “−” = no growth (N = 3).

| | CDem_D2 present | | | | | | | | | | | | | | | | | |
|---|---|---|---|---|---|---|---|---|---|---|---|---|---|---|---|---|---|---|
| | Clinell ® sporicidal | | | | | | | | | | | | | | | | | |
| In-test | solution | | | | | | EndoSan ® | | | | | | Chemgene ™ | | | | | |
| concen- | N = 1 | | N = 2 | | N = 3 | | N = 1 | | N = 2 | | N = 3 | | N = 1 | | N = 2 | | N = 3 | |
| tration (%) | 1.1 | 1.2 | 2.1 | 2.2 | 3.1 | 3.2 | 1.1 | 1.2 | 2.1 | 2.2 | 3.1 | 3.2 | 1.1 | 1.2 | 2.1 | 2.2 | 3.1 | 3.2 |
| 50 | + | + | + | + | + | + | + | + | + | + | + | + | + | + | + | + | + | + |
| 25 | + | + | + | + | + | + | + | + | + | + | + | + | + | + | + | + | + | + |
| 12.5 | + | + | + | + | + | + | + | + | + | + | + | + | + | + | + | + | + | + |
| 6.25 | + | + | + | + | + | + | + | + | + | + | + | + | + | + | + | + | + | + |
| 3.125 | + | + | + | + | + | + | + | + | + | + | + | + | + | + | + | + | + | + |
| 1.5625 | + | + | + | + | + | + | + | + | + | + | + | + | + | + | + | + | + | + |

TABLE 17
minimal sporicidal concentration (MSC) of Clinell ® sporicidal
solution, EndoSan ® and Chemgene ™ against
Clostridium difficile SH11 spores, in the presence and absence
of CotE_H2 aptamer.

| | Clinell ® | | |
|---|---|---|---|
| | sporicidal | MSC (%) | |
| Test Details | solution | EndoSan ® | Chemgene ™ |
| CotE_H2 absent | 25 | >50 | >50 |
| CotE-H2 present | >50 | >50 | >50 |

TABLE 18
Minimal sporicidal concentration (MSC) of Clinell ® sporicidal
solution, EndoSan ® and Chemgene ™ against
Clostridium difficile ATCC ® 43598 ™ spores, in the presence
and absence of CotE_H2, CDec_D1 and CDem_D2 aptamers.
* = no recovery on some replicates.

| | Clinell ® | | |
|---|---|---|---|
| | sporicidal | MSC % | |
| Test Details | solution | EndoSan ® | Chemgene ™ |
| Aptamers absent | >50* | >50 | >50 |
| CotE_H2 present | 50 | >50 | >50 |
| CDec_D1 present | 50 | >50 | >50 |
| CDem_D2 present | 25 | >50 | >50 |

### Discussion

When the test agents were assessed against C. difficile ATCC® 43598™ spores without the presence of the aptamers, a greater than 50% concentration was reported for all the test items. When C. difficile ATCC® 43598™ spores were tested in the presence of the Cote_H2 and CDec_D1 aptamers, the MSC for Clinell® sporicidal solution decreased. When C. difficile ATCC® 43598™ spores were tested in the presence of the CDem_D2 aptamer, a greater than two-fold decrease in the MSC was observed for Clinell® sporicidal solution. No change in the MSC was observed for EndoSan® and Chemgene™ in the presence of any aptamers.

When the test agents were assessed against C. difficile SH11 spores without the presence of aptamers, the lowest MSC (25%) was observed for Clinell® sporicidal solution. A greater than 50% concentration was observed for the MSC of EndoSan® and Chemgene™. When C. difficile SH11 spores were tested in the presence of the CDec_D1 aptamer, no change in the MSC was observed for any of the test items. When C. difficile SH11 spores were tested in the presence of the CDem_D2 aptamer, a greater than two-fold increase in the MSC was reported for Clinell® sporicidal solution (Table 19). No change in the MSC was observed for EndoSan® and Chemgene™.

TABLE 19
Minimal sporicidal concentration (MSC) of Clinell ® sporicidal
solution, EndoSan ® and Chemgene ™
against Clostridium difficile SH11 spores, in the presence and absence
of CotE_H2, CDec_D1 and Cdem_D2 aptamers.

| | Clinell ® | | |
|---|---|---|---|
| | sporicidal | MSC (%) | |
| Test Details | solution | EndoSan ® | Chemgene ™ |
| Aptamers absent | 25 | >50 | >50 |
| CDec_D1 present | 25 | >50 | >50 |
| CDem_D2 present | >50 | >50 | >50 |

### Discussion

Clostridium difficile is a spore-forming, Gram-positive obligative anaerobic bacterium. C. difficile infections are considered a leading cause of infections world-wide with increased rates of associated mortality and morbidity. The spores formed by C. difficile on surfaces continue to be viable for several weeks, facilitating the spread and occurrence of C. difficile infections. Sporicidal solutions have been designed to eradicate resilient spores and are regularly used to decontaminate surfaces in clinical settings. Aptamers are short, artificial single-stranded oligonucleotides comprising DNA and/or RNA which bind to their targets with high selectivity and sensitivity.

Following treatment for 15 minutes in the absence of aptamers, none of the test solutions demonstrated sporicidal activity against C. difficile ATCC® 43598™ spores at concentrations of ≤50%, whereas a concentration of 25% Clinell® sporicidal solution demonstrated sporicidal activity against C. difficile SH11 spores. In the presence of aptamers, the MSC of the Clinell® sporicidal solution decreased against C. difficile ATCC® 43598™ spores. However, the MSC of the Clinell® sporicidal solution increased more than two-fold against C. difficile SH11 spores. No change in the sporicidal solution of the EndoSan® and Chemgene™ solutions were observed against either strain of C. difficile spores. The results suggest that the aptamers may have different binding affinities for different strains of C. difficile spores.

### Example 3

The Clostridioides difficile (formerly Clostridium difficile) spore suspensions used in this study are listed in Table 20 below. Clostridioides difficile R20291 suspension was provided by SporeGen® on 23 Jan. 2020 and stored at 4 °C upon arrival. Clostridioides difficile SH11 suspension was provided by SporeGen® on 17 Feb. 2020 and stored at 4 °C upon arrival.

TABLE 20

| | | In-test |
|---|---|---|
| Ribotype | Description | Concentration |
| RT027 (R20291) | Purified spore | 1 × 10⁸ ± 5 × 10⁷ |
| | suspension | |
| RT078 (SH11) | Purified spore | 1 × 10⁸ ± 5 × 10⁷ |
| | suspension | |
| ATCC ® 43598 ™ | Internal strain | 1 × 10⁸ ± 5 × 10⁷ |
| | spore suspension | |

### Test Agents

Test agents used in this study are listed in Table 21 and Table 22 below. Aptamers and buffers were provided by Aptamer Group Limited.

TABLE 21

| | | Concentration | In-test |
|---|---|---|---|
| | | Provided | concentration |
| Test Agent Name | Buffer | (μM) | (μM) |
| CDiff_F1 aptamer | BB+ | 100 | 20, 10, 5, 2.5 |
| | Mg2+ | | |

TABLE 22

| | | | In-test |
|---|---|---|---|
| | | | concentration |
| Test Agent Name | Product Type | Lot Number | (μM) |
| Clinell sporicidal | Liquid sporicidal | SA613818 | 50, 25, 12.5, 6.25, |
| solution | | | 3.125, 1.5625 |
| Negative control | PBS + 1% RCM | PBS200109/ | Not applicable |
| | | RCM200109 | |
| Positive Control | Bleach based | 9 302 2696 | Not applicable |
| | solution | | |

### Equipment

UKAS calibrated pipettes (0.5-1000 μL range), Proline® Plus—Sartorius, UK
UKAS calibrated multichannel pipettes (P300 and P20)—Gilson®, UK
Eppendorf 5452 minispin centrifuge—Eppendorf, DE
Calibrated balance, Ohaus NV212—Scientific Laboratory Supplies Ltd (SLS), UK
Anaerobic cabinet, Whitley MG500—Don Whitley Scientific Limited, UK
Digital dry bath—SLS, UK
Sterile universal tubes—SLS, UK
96-well plates—SLS, UK

### Media

Nuclease free water—Aptamer Group, UK
BB+Mg2+ buffer—Aptamer Group, UK
Brain heart infusion agar—SLS, UK
Sodium taurocholate—Sigma-Aldrich, UK
Yeast extract—Sigma-Aldrich, UK
Horse blood—TCS Biosciences, UK
Reinforced clostridial medium (RCM), Acumedia®—SLS, UK
Phosphate buffered saline (PBS)—SLS, UK
Saponin—Sigma-Aldrich, UK
Sodium thiosulfate—Sigma-Aldrich, UK
Sodium dodecyl sulphate—Sigma-Aldrich, UK
Lecithin—Sigma-Aldrich, UK
L-histidine—Sigma-Aldrich, UK
Tween 80—Sigma-Aldrich, UK

Preparation of brain heart infusion agar supplemented with horse blood and yeast (BHI-YHT).

| Compound | Quantity | |
|---|---|---|
| Brain heart infusion agar | 52 | g |
| Yeast extract | 5 | g |
| Sodium taurocholate hydrate | 1 | g |
| Defibrinated horse blood | 70 | mL |
| Distilled water | 1 | L |

### Method

The Evaluation of the Sporicidal Enhancement Capabilities of the CDiff_F1 Aptamer in Combination with Clinell® Sporicidal Solution Against Clostridioides difficile Spores.

Prior to testing, the CDiff_F1 aptamers were diluted in nuclease free water to a concentration of 80 μM. The aptamers were folded by heating to 95 °C in a digital dry bath for 5 minutes and then immediately placed on ice and cooled to 2 °C. The aptamers were stored at 2-4 °C while in use. A C. difficile SH11 bacterial spore inoculum was prepared to 1×108±5×107 CFUmL−1 according to internal procedures. The inoculum was confirmed by serial dilution and plating out the resultant suspensions onto brain heart infusion agar supplemented with horse blood and yeast (BHI-YHT). The CDiff_F1 aptamers were further diluted in “BB+Mg2+” buffer to obtain two times in-test concentrations (40, 20, 10 and 5 μM). Ten microlitres of C. difficile spores were added to 10 μL of each two times in-test concentration of CDiff_F1 aptamer. The spore suspension was pipette mixed and incubated with CDiff_F1 aptamer at room temperature for one hour. The spores were then washed three times by centrifuging at 13,400 RPM for 10 minutes. In each wash the supernatant was removed, and the pellet was resuspended in 100 μL of “BB+Mg2+” buffer. Following the third wash, the spores were resuspended in 100 μL of “BB+Mg2+” buffer and vortexed to obtain a homogenous solution.

A total of six, two times in-test concentrations of each sporicidal test agent were prepared from the stock (100%, 50%, 25%, 12.5%, 6.25%, 3.125%). Negative and positive control solutions were also prepared. Ten microliter aliquots of each sporicidal test agent concentration or control solution were added to the wells of a 96-well plate. A 10 μL aliquot of the aptamer-spore suspension was added to each test agent or control solution and pipette mixed. The final in-test concentrations of each sporicidal test agent were 50%, 25%, 12.5%, 6.25%, 3.125%, 1.5625%. The plates were then incubated for 15 minutes at 37 °C±2 °C in an anaerobic environment. Following incubation, 50 μL of GH neutraliser was added to each well and pipette mixed. A 10 μL aliquot of neutralised suspension was taken from each well and transferred to an agar plate and incubated for 48 hours at 37 °C±2 °C in an anaerobic environment. Growth was recorded as “+” and no growth as “−”. The minimum sporicidal concentration (MSC) for each concentration was identified as the lowest concentration with no growth for all replicates. If growth was observed at the highest concentration it was displayed as “>”. The test was repeated without the presence of the aptamers and without the presence of the Clinell® sporicidal solution. Testing was also repeated using C. difficile ATCC® 43598™ and C. difficile R20291 spores. Testing was performed in triplicate.

### Results

The evaluation of the sporicidal enhancement capabilities of the CDiff_F1 aptamer in combination with Clinell® sporicidal solution against Clostridioides difficile spores

3.1.1 Clostridioides difficile SH11 Spores

The growth of C. difficile SH11 spores was unaffected by the presence of any concentration of CDiff_F1 aptamers without the presence of Clinell® sporicidal solution. When the six concentrations of Clinell® sporicidal solution were assessed against C. difficile SH11 spores without the presence of the CDiff_F1 aptamer, the MSC was >50%. In the presence of and 2.5 μM, 5 μM and 10 μM CDiff_F1 aptamers, the MSC of Clinell® sporicidal solution was 25%. This was a >2-fold decrease in the MSC of Clinell® sporicidal solution. In the presence of 20 μM CDiff_F1 aptamers, the MSC of Clinell® sporicidal solution was 12.5%. This was a >4-fold decrease in the MSC of Clinell® sporicidal solution.

TABLE 23
Minimal sporicidal concentration (MSC) of
CDiff_F1 aptamers and Clinell sporicidal solution
against C. difficile SH11 spores in the presence
and absence of various concentrations of CDiff_F1 aptamers.

| | | | Sporicidal |
|---|---|---|---|
| Concentration of | MSC of | MSC of Clinell | solution (%) |
| CDiff_F1 (μM) | CDiff_F1 (μM) | No. CDiff_F1 | with CDiff_F1 |
| 2.5 | >20 | >50 | 25 |
| 5 | >20 | >50 | 25 |
| 10 | >20 | >50 | 25 |
| 20 | >20 | >50 | 12.5 |

The growth of C. difficile ATCC® 43598™ spores was unaffected by the presence of any concentration of CDiff_F1 aptamers without the presence of Clinell® sporicidal solution (Table 24). When the six concentrations of Clinell® sporicidal solution were assessed against C. difficile ATCC® 43598 ™ spores without the presence of the CDiff_F1 aptamers, the MSC was >50%. In the presence of all the concentrations of CDiff_F1 aptamers, the MSC of Clinell® sporicidal solution was 25%. This was a >2-fold decrease in the MSC of Clinell® sporicidal solution.

TABLE 24
Minimal sporicidal concentration (MSC) of CDiff_F1 aptamers
and Clinell sporicidal solution against C. difficile
ATCC 43598 spores in the presence and absence of various
concentrations of CDiff_F1 aptamers.

| | | | Sporicidal |
|---|---|---|---|
| Concentration of | MSC of | MSC of Clinell | solution (%) |
| CDiff_F1 (μM) | CDiff_F1 (μM) | No. CDiff_F1 | with CDiff_F1 |
| 2.5 | >20 | >50* | 25 |
| 5 | >20 | >50* | 25 |
| 10 | >20 | >50* | 25 |
| 20 | >20 | >50* | 25 |

*= No growth observed for one replicate showing that 50% Clinell sporicidal solution was enough to kill all spores for one replicate, however, 50% cannot be considered the MSC as growth was observed for the other two replicates.

TABLE 25
Minimal sporicidal concentration (MSC) of
CDiff_F1 aptamers and Clinell sporicidal solution
against C. difficile R20291 spores in the presence
and absence of various concentrations of CDiff_F1 aptamers.

| | | | Sporicidal |
|---|---|---|---|
| Concentration of | MSC of | MSC of Clinell | solution (%) |
| CDiff_F1 (μM) | CDiff_F1 (μM) | No. CDiff_F1 | with CDiff_F1 |
| 2.5 | >20 | >50 | >50 |
| 5 | >20 | >50 | >50 |
| 10 | >20 | >50 | >50 |
| 20 | >20 | >50 | 50 |

TABLE 26
Sporicidal concentrations of Clinell ® sporicidal solution
in the presence and absence of four concentrations of CDiff_F1 aptamers
using C. difficile SH11 spores. Growth observed after recovering
viable C. difficile SH11 spores following treatment for 15 minutes with
six concentrations of Clinell sporicidal solution in the absence of
CDiff_F1 aptamers “+” = growth present, “−” =
no growth (n = 3).

| Concentration | | | |
|---|---|---|---|
| of Clinell ® | Aptamers absent | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | + | + | + | + | + | + |
| 25 | + | + | + | + | + | + |
| 12.5 | + | + | + | + | + | + |
| 6.25 | + | + | + | + | + | + |
| 3.125 | + | + | + | + | + | + |
| 1.5625 | + | + | + | + | + | + |

TABLE 27
Growth observed after recovering viable C. difficile
SH11 spores following treatment for 15 minutes with six concentrations
of Clinell ® sporicidal solution in the presence of CDiff_F1 aptamers
at 20 μM “+” = growth present, “−” = no growth (n = 3).

| Concentration | | | |
|---|---|---|---|
| of Clinell ® | CDiff_F1 (20 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | − | − | − | − | − | − |
| 6.25 | + | + | + | + | + | + |
| 3.125 | + | + | + | + | + | + |
| 1.5625 | + | + | + | + | + | + |

TABLE 28
Growth observed after recovering viable C. difficile
SH11 spores following treatment for 15 minutes with six concentrations
of Clinell ® sporicidal solution in the presence of CDiff_F1 aptamers
at 10 μM “+” = growth present, “−” = no growth (n = 3).

| Concentration | | | |
|---|---|---|---|
| of Clinell ® | CDiff_F1 (10 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | − | − | − | − | + | + |
| 6.25 | + | + | + | + | + | + |
| 3.125 | + | + | + | + | + | + |
| 1.5625 | + | + | + | + | + | + |

TABLE 29
Growth observed after recovering viable C. difficile
SH11 spores following treatment for 15 minutes with six concentrations
of Clinell ® sporicidal solution in the presence of CDiff_F1 aptamers
at 5 μM “+” = growth present, “−” = no growth (n = 3).

| Concentration | | | |
|---|---|---|---|
| of Clinell ® | CDiff_F1 (5 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | − | − | + | + | + | + |
| 6.25 | + | + | + | + | + | + |
| 3.125 | + | + | + | + | + | + |
| 1.5625 | + | + | + | + | + | + |

TABLE 30
Growth observed after recovering viable C. difficile
SH11 spores following treatment for 15 minutes with six concentrations
of Clinell ® sporicidal solution in the presence of CDiff_F1 aptamers
at 2.5 μM “+” = growth present, “−” = no growth (n = 3).

| Concentration | | | |
|---|---|---|---|
| of Clinell ® | CDiff_F1 (2.5 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | + | + | + | + | + | + |
| 6.25 | + | + | + | + | + | + |
| 3.125 | + | + | + | + | + | + |
| 1.5625 | + | + | + | + | + | + |

TABLE 31
Growth observed after recovering viable C. difficile SH11
spores following treatment for 15 minutes with four concentrations
of aptamer “+” = growth present, “−” = no growth (n = 3).

| Concentration | Aptamers only | | |
|---|---|---|---|
| of CDiff_F1 | N = 1 | N = 2 | N = 3 |

| aptamer (μM) | 1.1 | 1.2 | 2.1 | 2.2 | 3.1 | 3.2 |
|---|---|---|---|---|---|---|
| 20 | + | + | + | + | + | + |
| 10 | + | + | + | + | + | + |
| 5 | + | + | + | + | + | + |
| 2.5 | + | + | + | + | + | + |

TABLE 32
Sporicidal concentrations of Clinell ® sporicidal solution in the
absence of CDiff_F1 aptamers using C. difficile ATCC 43598 spores.
Growth observed after recovering viable C. difficile 43598 spores
following treatment for 15 minutes with six concentrations of
Clinell sporicidal solution in the absence of CDiff_F1 aptamers
“+” = growth present, “−” = no growth (n = 3).

| Concentration | | | |
|---|---|---|---|
| of Clinell ® | Aptamers Absent | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | + | + | + | + |
| 25 | + | + | + | + | + | + |
| 12.5 | + | + | + | + | + | + |
| 6.25 | + | + | + | + | + | + |
| 3.125 | + | + | + | + | + | + |
| 1.5625 | + | + | + | + | + | + |

TABLE 33
Sporicidal concentrations of Clinell ® sporicidal solution
in the presence and absence of four concentrations of CDiff_F1
aptamers using C. difficile ATCC 43598 spores. Growth observed
after recovering viable C. difficile 43598 spores following treatment
for 15 minutes with six concentrations of Clinell sporicidal solution
in the presence of CDiff_F1 aptamers at 20 μM “+” =
growth present, “−” = no growth (n = 3).

| Concentration | | | |
|---|---|---|---|
| of Clinell ® | CDiff_F1 (20 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | + | + | + | + | + | + |
| 6.25 | + | + | + | + | + | + |
| 3.125 | + | + | + | + | + | + |
| 1.5625 | + | + | + | + | + | + |

TABLE 34
Sporicidal concentrations of Clinell ® sporicidal solution
in the presence and absence of four concentrations of CDiff_F1
aptamers using C. difficile ATCC 43598 spores. Growth observed
after recovering viable C. difficile 43598 spores following treatment
for 15 minutes with six concentrations of Clinell ® sporicidal
solution in the presence of CDiff_F1 aptamers at 10 μM + =
growth present, − = no growth (n = 3).

| Concentration | | | |
|---|---|---|---|
| of Clinell ® | CDiff_F1 (10 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | + | + | + | + | + | + |
| 6.25 | + | + | + | + | + | + |
| 3.125 | + | + | + | + | + | + |
| 1.5625 | + | + | + | + | + | + |

TABLE 35
Sporicidal concentrations of Clinell ® sporicidal solution in the
presence and absence of four concentrations of CDiff_F1 aptamers using
C. difficile ATCC 43598 spores. Growth observed after recovering viable
C. difficile 43598 spores following treatment for 15 minutes with six
concentrations of Clinell ® sporicidal solution in the presence of
CDiff_F1 aptamers at 5 μM + = growth present, − = no growth (n = 3).

| Concentration | | | |
|---|---|---|---|
| of Clinell ® | CDiff_F1 (5 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | + | + | + | + | + | + |
| 6.25 | + | + | + | + | + | + |
| 3.125 | + | + | + | + | + | + |
| 1.5625 | + | + | + | + | + | + |

TABLE 36
Sporicidal concentrations of Clinell ® sporicidal solution
in the presence and absence of four concentrations of CDiff_F1
aptamers using C. difficile ATCC 43598 spores. Growth observed
after recovering viable C. difficile 43598 spores following treatment
for 15 minutes with six concentrations of Clinell ® sporicidal
solution in the presence of CDiff_F1 aptamers at 2.5 μM + =
growth present, − = no growth (n = 3).

| Concentration | | | |
|---|---|---|---|
| of Clinell ® | CDiff_F1 (2.5 μM) | | |
| sporicidal | N = 1 | N = 2 | N = 3 |

| solution (%) | 1.1 | 1.2 | 2.1 | 2.2 | 3.1 | 3.2 |
|---|---|---|---|---|---|---|
| 50 | − | − | − | − | − | − |
| 25 | − | − | − | − | − | − |
| 12.5 | + | + | + | + | + | + |
| 6.25 | + | + | + | + | + | + |
| 3.125 | + | + | + | + | + | + |
| 1.5625 | + | + | + | + | + | + |

TABLE 37
Growth observed after recovering viable C. difficile ATCC 43598
spores following treatment for 15 minutes with four concentrations
of aptamer “+” = growth present, “−” = no growth (n = 3).

| Concentration | Aptamers only | | | | | |
|---|---|---|---|---|---|---|
| of CDiff_F1 | N = 1 | | N = 2 | | N = 3 | |
| aptamer (μM) | 1.1 | 1.2 | 2.1 | 2.2 | 3.1 | 3.2 |
| 20 | + | + | + | + | + | + |
| 10 | + | + | + | + | + | + |
| 5 | + | + | + | + | + | + |
| 2.5 | + | + | + | + | + | + |

TABLE 38
Sporicidal concentrations of Clinell ® sporicidal solution in
the presence and absence of four concentrations of CDiff_F1
aptamers using C. difficile R20291 spores. Growth observed
after recovering viable C. difficile R20291 spores following
treatment for 15 minutes with six concentrations of Clinell
sporicidal solution in the absence of CDiff_F1
aptamers “+” = growth present, “−” = no growth (n = 3).

| Concentration | | | | | | |
|---|---|---|---|---|---|---|
| of Clinell ® | Aptamers Absent | | | | | |
| sporicidal | N = 1 | | N = 2 | | N = 3 | |
| solution (%) | 1.1 | 1.2 | 2.1 | 2.2 | 3.1 | 3.2 |
| 50 | + | + | + | + | + | + |
| 25 | + | + | + | + | + | + |
| 12.5 | + | + | + | + | + | + |
| 6.25 | + | + | + | + | + | + |
| 3.125 | + | + | + | + | + | + |
| 1.5625 | + | + | + | + | + | + |

TABLE 39
Sporicidal concentrations of Clinell ® sporicidal solution in the
presence and absence of four concentrations of CDiff_F1
aptamers using C. difficile R20291 spores. Growth observed
after recovering viable C. difficile R20291 spores following
treatment for 15 minutes with six concentrations of Clinell ®
sporicidal solution in the presence of CDiff_F1 aptamers at
20 μM “+” = growth present, “−” = no growth (n = 3).

| Concentration | CDiff_F1 (20 μM) | | | | | |
|---|---|---|---|---|---|---|
| of Clinell ® | N = 1 | | N = 2 | | N = 3 | |
| sporicidal solution (%) | 1.1 | 1.2 | 2.1 | 2.2 | 3.1 | 3.2 |
| 50 | − | − | − | − | − | − |
| 25 | − | + | + | + | − | − |
| 12.5 | + | + | + | + | + | + |
| 6.25 | + | + | + | + | + | + |
| 3.125 | + | + | + | + | + | + |
| 1.5625 | + | + | + | + | + | + |

TABLE 40
Sporicidal concentrations of Clinell ® sporicidal solution in the
presence and absence of four concentrations of
CDiff_F1 aptamers using C. difficile R20291 spores. Growth observed
after recovering viable C. difficile R20291 spores following
treatment for 15 minutes with six concentrations of Clinell ® sporicidal
solution in the presence of CDiff_F1 aptamers at
10 μM “+” = growth present, “−” = no growth (n = 3).

| Concentration | | | | | | |
|---|---|---|---|---|---|---|
| of Clinell ® | CDiff_F1 (10 μM) | | | | | |
| solution (%) | N = 1 | | N = 2 | | N = 3 | |
| sporicidal | 1.1 | 1.2 | 2.1 | 2.2 | 3.1 | 3.2 |
| 50 | − | + | + | + | − | − |
| 25 | + | + | + | + | + | + |
| 12.5 | + | + | + | + | + | + |
| 6.25 | + | + | + | + | + | + |
| 3.125 | + | + | + | + | + | + |
| 1.5625 | + | + | + | + | + | + |

TABLE 41
Sporicidal concentrations of Clinell ® sporicidal solution in the
presence and absence of four concentrations of CDiff_F1
aptamers using C. difficile R20291 spores. Growth observed after
recovering viable C. difficile R20291 spores following
treatment for 15 minutes with six concentrations of Clinell ®
sporicidal solution in the presence of CDiff_F1 aptamers at
5 μM “+” = growth present, “−” = no growth (n = 3).

| Concentration | CDiff_F1 (5 μM) | | | | | |
|---|---|---|---|---|---|---|
| of Clinell ® sporicidal | N = 1 | | N = 2 | | N = 3 | |
| solution (%) | 1.1 | 1.2 | 2.1 | 2.2 | 3.1 | 3.2 |
| 50 | + | + | + | + | + | + |
| 25 | + | + | + | + | + | + |
| 12.5 | + | + | + | + | + | + |
| 6.25 | + | + | + | + | + | + |
| 3.125 | + | + | + | + | + | + |
| 1.5625 | + | + | + | + | + | + |

TABLE 42
Sporicidal concentrations of Clinell ® sporicidal solution in the
presence and absence of four concentrations of CDiff_F1
aptamers using C. difficile R20291 spores. Growth observed
after recovering viable C. difficile R20291 spores following
treatment for 15 minutes with six concentrations of Clinell ®
sporicidal solution in the presence of CDiff_F1 aptamers at
5 μM “+” = growth present, “−” = no growth (n = 3).

| Concentration | CDiff_F1 (2.5 μM) | | | | | |
|---|---|---|---|---|---|---|
| of Clinell ® | N = 1 | | N = 2 | | N = 3 | |
| sporicidal solution (%) | 1.1 | 1.2 | 2.1 | 2.2 | 3.1 | 3.2 |
| 50 | + | + | + | + | + | + |
| 25 | + | + | + | + | + | + |
| 12.5 | + | + | + | + | + | + |
| 6.25 | + | + | + | + | + | + |
| 3.125 | + | + | + | + | + | + |
| 1.5625 | + | + | + | + | + | + |

TABLE 43
Growth observed after recovering viable C. difficile R20291 spores
following treatment for 15 minutes with four concentrations of
aptamer “+” = growth present, “−” = no growth (n = 3).

| Concentration | Aptamers only | | | | | |
|---|---|---|---|---|---|---|
| of CDiff_F1 | N = 1 | | N = 2 | | N = 3 | |
| aptamer (μM) | 1.1 | 1.2 | 2.1 | 2.2 | 3.1 | 3.2 |
| 20 | + | + | + | + | + | + |
| 10 | + | + | + | + | + | + |
| 5 | + | + | + | + | + | + |
| 2.5 | + | + | + | + | + | + |

### Discussion

C. difficile is an anaerobic spore-forming bacterium recognised for causing nosocomial infections worldwide. C. difficile spores exist in a wide range of environments due to their resilience to cleaning regimes. Sporicidal solutions have been designed to eradicate resilient spores and are regularly used to decontaminate surfaces in clinical settings. The addition of CDiff_F1 aptamers enhanced the sporicidal capabilities of the Clinell® sporicidal solution against all three strains of C. difficile spores. The sporicidal enhancement of the Clinell® sporicidal solution varied between the three strains, with the greatest reduction in the MSC in the Clinell® sporicidal solution observed from C. difficile SH11 spores in combination with 20 μM CDiff_F1 aptamers. The addition of only CDiff_F1 aptamers did not affect the growth of any strain of C. difficile spores which may suggest an interaction between the aptamers and the Clinell® sporicidal solution once bound to the spores. The results suggest that CDiff_F1 aptamer may have the greatest binding affinity for C. difficile SH11 spores.

### Example 4

### Test Microorganisms

The Clostridioides difficile (formerly Clostridium difficile) spore suspensions used in this study are listed in Table 1. Clostridioides difficile R20291 suspension was provided by SporeGen® on 23 Jan. 2020 and stored at 4 °C upon arrival. Clostridioides difficile SH11 suspension was provided by SporeGen® on 17 Feb. 2020 and stored at 4 °C upon arrival.

TABLE 44
C. difficile strains

| | | | In-test |
|---|---|---|---|
| | | | concentration |
| Organism | Ribotype | Description | (CFUmL-1) |
| C. difficile | RT027 (R20291) | Purified spore suspension | 1 × 10⁷ ± 5 × 10⁶ |
| C. difficile | RT078 (SHU) | Purified spore suspension | 1 × 10⁷ ± 5 × 10⁶ |
| C. difficile | ATCC ® 43598 ™ | Internal strain spore suspension | 1 × 10⁷ ± 5 × 10⁶ |

TABLE 45
Test Agents Used

| | | Lot | In-test |
|---|---|---|---|
| Test agent name | Product type | number | concentrations (%) |
| Clinell ® sporicidal | Liquid | SA613818 | 50, 25, 12.5, 6.25, |
| solution | sporicidal | | 3.125, 1.5625 |
| Negative control | PBS + 1% | PBS200122/ | N/A |
| | RCM | RCM200109 | |
| Positive control | Bleach-based | 9 302 2696 | N/A |
| | solution | | |

PBS = phosphate buffered saline,
RCM = reinforced clostridial medium.

### Equipment and Media

### Media

Nuclease free water—provided by Aptamer Group, UK
“TbKst” buffer—provided by Aptamer group, UK “TbKat” buffer—provided by
Aptamer group, UK Sodium taurocholate—Sigma-Aldrich, UK
Yeast extract—Sigma-Aldrich, UK Horse blood—TCS Biosciences Ltd, UK
Reinforced clostridial medium (RCM), Acumedia®—SLS, UK Phosphate buffered
saline (PBS)—SLS, UK
Saponin—Sigma-Aldrich, UK
Sodium thiosulfate—Sigma-Aldrich, UK Sodium dodecyl sulphate—Sigma-Aldrich, UK Lecithin—Sigma-Aldrich, UK
L-histidine—Sigma-Aldrich, UK Tween 80—Sigma-Aldrich, UK

### Method

Evaluation of the Sporicidal Enhancement Capabilities of CotE_H2 and CDec_D1 Aptamers in Combination with Clinell® Sporicidal Solution Against Clostridioides difficile Spores.

Prior to testing, CotE_H2 aptamers were diluted in nuclease free water to a concentration of 80 μM. The aptamers were folded by heating to 95 °C in a digital dry bath for 5 minutes and then immediately placed on ice and cooled to 2 °C. The aptamers were stored at 2-4 °C while in use. A Clostridioides difficile SH11 bacterial spore inoculum was prepared to 1×10⁷±5×10⁶ CFUmL⁻¹ according to internal procedures. The inoculum was confirmed by serial dilution and plating out the resultant suspensions onto brain heart infusion agar supplemented with horse blood and yeast (BHI-YHT). CotE_H2 aptamers were further diluted in “TbKst” buffer to obtain 2× in-test concentrations (40, 20, 10 and 5 μM). Ten microlitres of C. difficile spores were added to 10 μL of each 2× in-test concentration of CotE_H2 aptamer. The spore suspension was pipette mixed and incubated with CotE_H2 aptamer at room temperature for one hour. The spores were then washed three times by centrifuging at 13,400 RPM for 10 minutes. In each wash the supernatant was removed, and the pellet was resuspended in 100 μL of “TbKst” buffer. Following the third wash, the spores were resuspended in 100 μL of “TbKst” buffer and vortexed to obtain a homogenous solution.

A total of six 2× in-test concentrations (1:2 dilutions) of the sporicidal test agent were prepared from the stock (100%, 50%, 25%, 12.5%, 6.25%, 3.125%). Negative and positive control solutions were also prepared. Ten microliter aliquots of each sporicidal test agent concentration or control solution were added to the wells of a 96-well plate. A 10 μL aliquot of the aptamer-spore suspension was added to each test agent or control solution and pipette mixed. The final in-test concentrations of the sporicidal test agent were 50%, 25%, 12.5%, 6.25%, 3.125%, 1.5625%. The plates were then incubated for 15 minutes at 37 °C±2 °C in an anaerobic environment. Following incubation, 50 μL of GH neutraliser was added to each well and pipette mixed. A 10 μL aliquot of neutralised suspension was taken from each well and transferred to an agar plate and incubated for 48 hours at 37 °C±2 °C in an anaerobic environment. Growth was recorded as “+” and no growth as “−”. The minimum sporicidal concentration (MSC) for each spore and aptamer concentration was identified as the lowest concentration with no growth for all replicates. If growth was observed at the highest concentration it was displayed as “>”. The test was repeated without the presence of the aptamers and without the presence of the Clinell® sporicidal solution. Testing was also repeated using C. difficile ATCC® 43598™ and C. difficile R20291 spores. Testing was performed in triplicate. The method was repeated with CDec_D1 aptamers for all three strains of C. difficile spores.

### Results

Evaluation of the Sporicidal Enhancement Capabilities of the CotE_H2 Aptamer in Combination with Clinell® Sporicidal Solution Against Clostridioides difficile Spores.
Clostridioides difficile SH11 Spores

The growth of C. difficile SH11 spores was unaffected by the presence of any concentration of CotE_H2 aptamers without the presence of the Clinell® sporicidal solution. When the six concentrations of Clinell® sporicidal solution were assessed against C. difficile SH11 spores without the presence of the CotE_H2 aptamer, the MSC was 25%. In the presence of 20 μM, 10 μM, 5 μM and 2.5 μM CotE_H2 aptamers, the MSC of Clinell® sporicidal solution was 12.5%. This was a two-fold decrease in the MSC of Clinell® sporicidal solution.

TABLE 46
Minimal sporicidal concentration (MSC) of CotE_H2 aptamers
and Clinell ® sporicidal solution against Clostridioides difficile SH11
spores, in the presence and absence of various concentrations of
CotE_H2 aptamers.

| | MSC | MSC of Clinell ® sporicidal solution (%) | |
|---|---|---|---|
| Concentration of | of CotE_H2 | | With |
| CotE_H2 (μM) | (μM) | No CotE_H2 | CotE_H2 |
| 2.5 | >20 | 25 | 12.5 |
| 5 | >20 | 25 | 12.5 |
| 10 | >20 | 25 | 12.5 |
| 20 | >20 | 25 | 12.5 |

Clostridioides difficile ATCC® 43598™ Spores

The growth of C. difficile ATCC® 43598™ spores was unaffected by the presence of any concentration of CotE_H2 aptamers without the presence of the Clinell® sporicidal solution. When the six concentrations of Clinell® sporicidal solution were assessed against C. difficile ATCC® 43598™ spores without the presence of the CotE_H2 aptamers, the MSC was 12.5%. The presence of 20 μM, 10 μM, 5 μM and 2.5 μM CotE_H2 aptamers did not affect the MSC of Clinell® sporicidal solution.

TABLE 47
Minimal sporicidal concentration (MSC) of CotE_H2 aptamers and
Clinell ® sporicidal solution against Clostridioides difficile ATCC ®
43598 ™ spores, in the presence and absence of various
concentrations of CotE_H2 aptamers.

| Concentration | MSC of | MSC of Clinell ® sporicidal | |
|---|---|---|---|
| of | CotE_H2 | solution (%) | |
| CotE_H2 (μM) | (μM) | No CotE_H2 | With CotE_H2 |
| 2.5 | >20 | 12.5 | 12.5 |
| 5 | >20 | 12.5 | 12.5 |
| 10 | >20 | 12.5 | 12.5 |
| 20 | >20 | 12.5 | 12.5 |

Clostridioides difficile R20291 Spores

The growth of C. difficile R20291 spores was unaffected by the presence of any concentration of CotE_H2 aptamers without the presence of the Clinell® sporicidal solution. When the six concentrations of Clinell® sporicidal solution were assessed against C. difficile R20291 spores without the presence of the CotE_H2 aptamers, the MSC was 25%. In the presence of 20 μM CotE_H2 aptamers, the MSC of Clinell® sporicidal solution was 12.5%. This was a two-fold decrease in the MSC of Clinell® sporicidal solution. The presence of 10 μM, 5 μM and 2.5 μM CotE_H2 aptamers did not affect the MSC of Clinell® sporicidal solution when assessed against C. difficile R20291 spores.

TABLE 48
Minimal sporicidal concentration (MSC) of Clinell ® sporicidal solution
against Clostridioides difficile R20291 spores, in the presence
and absence of various concentrations of CotE_H2 aptamers.

| | MSC | MSC of | |
|---|---|---|---|
| Concentration of | of CotE_H2 | Clinell® sporicidal solution | |
| CotE_H2 (μM) | (μM) | No CotE_H2 | With CotE_H2 |
| 2.5 | >20 | 25 | 25 |
| 5 | >20 | 25 | 25 |
| 10 | >20 | 25 | 25 |
| 20 | >20 | 25 | 12.5 |

### Example 6

Evaluation of the sporicidal enhancement capabilities of the CDec_D1 aptamer in combination with Clinell® sporicidal solution against Clostridioides difficile spores

Clostridioides difficile SH11 Spores

The growth of C. difficile SH11 spores was unaffected by the presence of any concentration of CDec_D1 aptamers without the presence of the Clinell® sporicidal solution. When the six concentrations of Clinell® sporicidal solution were assessed against C. difficile SH11 spores without the presence of the CDec_D1 aptamer, the MSC was 50%. In the presence of 20 μM, 10 μM and 5 μM CDec_D1 aptamers, the MSC of Clinell® sporicidal solution was 25%. This was a 2-fold decrease in the MSC of Clinell® sporicidal solution. The presence of 2.5 μM CDec_D1 aptamers did not affect the MSC of Clinell® sporicidal solution.

TABLE 49
Minimal sporicidal concentration (MSC) of Clinell ® sporicidal solution
against Clostridioides difficile SH11 spores, in the presence and absence
of various concentrations of CDec_D1 aptamers.

| Concentration of | MSC of | MSC of Clinell ® sporicidal | |
|---|---|---|---|
| CDec_D1 | CDec_D1 | solution (%) | |
| (μM) | (μM) | No CDec_D1 | With CDec_D1 |
| 2.5 | >20 | 50 | 50 |
| 5 | >20 | 50 | 25 |
| 10 | >20 | 50 | 25 |
| 20 | >20 | 50 | 25 |

Clostridioides difficile ATCC® 43598™ Spores

The growth of C. difficile ATCC® 43598™ spores was unaffected by the presence of any concentration of CDec_D1 aptamers without the presence of the Clinell® sporicidal solution. When the six concentrations of Clinell® sporicidal solution were assessed against C. difficile ATCC®43598™ spores without the presence of the CDec_D1 aptamers, the MSC was 25%. In the presence of 20 μM, 10 μM and 5 μM CDec_D1 aptamers, the MSC of Clinell® sporicidal solution was 12.5%. This was a two-fold decrease in the MSC of Clinell® sporicidal solution. The presence of 2.5 μM CDec_D1 aptamers did not affect the MSC of Clinell® sporicidal solution.

TABLE 50
Minimal sporicidal concentration (MSC) of Clinell ® sporicidal solution
against Clostridioides difficile ATCC ® 43598 ™ spores, in the presence
and absence of various concentrations of CDec_D1 aptamers.

| | MSC of | MSC of Clinell ® | |
|---|---|---|---|
| Concentration of | CDec_D1 | sporicidal solution (%) | |
| CDec_D1 (μM) | (μM) | No CDec_D1 | With CDec_D1 |
| 2.5 | >20 | 25 | 25 |
| 5 | >20 | 25 | 12.5 |
| 10 | >20 | 25 | 12.5 |
| 20 | >20 | 25 | 12.5 |

Clostridioides difficile R20291 Spores

The growth of C. difficile R20291 spores was unaffected by the presence of any concentration of CDec_D1 aptamers without the presence of the Clinell® sporicidal solution. When the six concentrations of Clinell® sporicidal solution were assessed against C. difficile R20291 spores without the presence of the CDec_D1 aptamers, the MSC was 50%. In the presence of 20 μM and 10 μM CDec_D1 aptamers, the MSC of Clinell® sporicidal solution was 12.5%. This was a 4-fold decrease in the MSC of Clinell® sporicidal solution. The presence of 5 μM and 2.5 μM CDec_D1 aptamers did not affect the MSC of Clinell® sporicidal solution when assessed against C. Difficile R20291 spores.

TABLE 51
Minimal sporicidal concentration (MSC) of Clinell ® sporicidal solution
against Clostridioides difficile R20291 spores, in the presence
and absence of various concentrations of CDec_D1 aptamers.

| | | MSC of Clinell ® sporicidal | |
|---|---|---|---|
| Concentration of | MSC of CDec_D1 | solution (%) | |
| CDec_D1 (μM) | (μM) | No CDec_D1 | With CDec_D1 |
| 2.5 | >20 | 50 | 50 |
| 5 | >20 | 50 | 50 |
| 10 | >20 | 50 | 12.5 |
| 20 | >20 | 50 | 12.5 |

### Discussion

Clostridioides difficile is an anaerobic spore-forming bacterium, recognised for causing nosocomial infections worldwide. Clostridioides difficile spores exist in a wide range of environments due to their resilience to cleaning regimes. Sporicidal solutions have been designed to eradicate resilient spores and are regularly used to decontaminate surfaces in clinical settings. The presence of an exosporium layer makes C. difficile spores more resistant to sporicidal products in comparison to other spore strains, as the sporicidal agents may struggle to penetrate this layer. Aptamers are short, artificial, single-stranded DNA or RNA oligonucleotides which bind to their targets with high selectivity and sensitivity.

The addition of CotE_H2 aptamers enhanced the sporicidal capabilities of the Clinell® sporicidal solution against C. difficile SH11 and R20291 spores. The addition of all concentrations of CotE_H2 aptamers reduced the MSC of the Clinell® sporicidal solution against C. difficile SH11 spores, whereas only the addition of 20 μM CotE_H2 aptamers reduced the MSC of the Clinell® sporicidal solution against C. difficile R20291 spores.

The addition of CDec_D1 aptamers enhanced the sporicidal capabilities of the Clinell® sporicidal solution against all three strains of C. difficile spores. The sporicidal enhancement varied between the three strains, with the greatest fold reduction observed in the MSC of the Clinell® sporicidal against C. difficile R20291 spores in combination with 10 μM and 20 μM CDec_D1 aptamers. Aptamers may affect the biochemical or biophysical properties of the exosporium layer of C. difficile spores during binding, which may increase their susceptibility to sporicidal products.

The results suggest that the CotE_H2 aptamers have the greatest affinity for the C. difficile SH11 spores, whereas the CDec_D1 aptamers have the greatest affinity for the C. difficile R20291 spores. The results also suggest that the sporicidal enhancement is dependent on the concentration of aptamers present as the addition of 2.5 μM CDec_D1 aptamers did not affect the MSC of the Clinell® sporicidal solution against all three strains of C. difficile spores.

### Discussion

Aptamers described herein may have utility in enhancing the sporicidal activity of a sporicidal agent such as peracetic acid. Different aptamers (raised to different targets of the C. difficile spore) may have different effects on the sporicidal activity of peracetic acid against different spore strains. For example, 20 μM of the CDiff_F1 aptamer may result in an up to 4-fold reduction in the concentration of peracetic acid required for sporicidal activity against spores of the SH11 strain. Furthermore, CDiff_F1 aptamer may result in an up to 2-fold reduction in the concentration of peracetic acid required for sporicidal activity against spores of the ATCC 43598 strain after 15 minutes. Aptamer Chitinase_D11 and Aptamer CdeC_D1 may each result in an up to 4-fold reduction in the concentration of peracetic acid required for sporicidal activity against spores of the R20291 strain.

The references cited throughout this application, are incorporated herein in their entireties for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this disclosure is not limited to the particular embodiments disclosed but is intended to cover all modifications which are within the spirit and scope of the disclosure as defined by the appended claims and/or the above description.

What is claimed is:

1. A kit for killing or inactivating C. difficile spores comprising:
an aptamer capable of specifically binding to C. difficile; and
a sporicidal agent.

2. The kit according to claim 1, wherein the aptamer comprises a nucleic acid sequence selected from any one of the nucleic acid sequences as set forth in any of SEQ ID NOs: 1 to 14, 23 to 39, or 43 to 55.

3. The kit according to claim 1, wherein the aptamer comprises a nucleic acid sequence having at least 90% sequence identity with any one of the nucleic acid sequences as set forth in any of SEQ ID NOs: 1 to 14, 23 to 39, or 43 to 55.

4. The kit according to claim 1, wherein the aptamer comprises a nucleic acid sequence having at least 95% sequence identity with any one of the nucleic acid sequences as set forth in any of SEQ ID NOs: 1 to 14, 23 to 39, or 43 to 55.

5. The kit according to claim 1, wherein the aptamer comprises a nucleic acid sequence having at least about 20 consecutive nucleotides of any one the nucleic acid sequences as set forth in any of SEQ ID NOs: 1 to 14, 23 to 39, or 43 to 55.

5. The kit according to claim 1, wherein the aptamer comprises at least about 20 consecutive nucleotides of a sequence having at least 85% identity with any one of SEQ ID NOs: 1 to 14, 23 to 39, or 43 to 55.

6. The kit according to claim 1, wherein the aptamer comprises at least one of:
a) an aptamer which specifically binds to a CdeC;
b) an aptamer which specifically binds to CdeM,
c) an aptamer which specifically binds to CotA,
d) an aptamer which specifically binds to CotE;
e) an aptamer which specifically binds to CotE Chitinase; or
f) a combination of any of (a) to (e).

7. The kit according to claim 1, comprising a plurality of aptamers comprising:
(a) a nucleic acid sequence selected from any one of the nucleic acid sequences as set forth in any of SEQ ID NOs: 1 to 14, 23 to 39, or 43 to 55;
(b) a nucleic acid sequence having at least 90% sequence identity with any one of the nucleic acid sequences as set forth in any of SEQ ID NOs: 1 to 14, 23 to 39, or 43 to 55;
(c) a nucleic acid sequence having at least about 20 consecutive nucleotides of any one the nucleic acid sequences as set forth in any of SEQ ID NOs: 1 to 14, 23 to 39, or 43 to 55;
(d) a nucleic acid sequence having at least about 20 consecutive nucleotides of a sequence having at least 85% identity with any one of SEQ ID NOs: 1 to 14, 23 to 39, or 43 to 55, or
(e) a combination of any of (a) to (d).

8. The kit according to claim 7, wherein the plurality of aptamers comprising:
a nucleic acid sequence as set forth in SEQ. ID. NO. 1;
a nucleic acid sequence as set forth in SEQ. ID. NO. 5; and
a nucleic acid sequence as set forth in SEQ. ID. NO. 6.

9. The kit according to claim 1, wherein the sporicidal agent comprises peracetic acid and/or an agent capable of generating peracetic acid.

10. The kit according to claim 1, wherein the aptamer enhances a sporicidal effect of the sporicidal agent against C. difficile spores.

11. The kit according to claim 1 further comprising:
a light source and viewing goggles for determining the presence, absence and/or concentration of C. difficile at a location prior to contacting the location with the sporicidal agent.

12. The kit of claim 1 further comprising instructions for use.

13. The kit of claim 12, wherein the instructions comprise locating the aptamer and the sporicidal agent at a location suspected of comprising C. difficile spores, contacting the location with the sporicidal agent prior to, at essentially the same time, or subsequent to contacting the location with the aptamer.

14. The kit of claim 13 wherein the contacting comprises submerging, spraying or applying the sporicidal agent by means of a cloth and/or a wipe.

15. A composition comprising one or more aptamers, wherein the one or more aptamers comprise one or more a nucleic acid sequence having at least 90% sequence identity with any one of the nucleic acid sequences as set forth in any of SEQ ID NOs: 1 to 14, 23 to 39, or 43 to 55.

16. The composition according to claim 15, wherein the one or more aptamers comprises a detectable label.

17. The composition according to claim 16, wherein the detectable label is selected from a fluorophore, a nanoparticle, a quantum dot, an enzyme, a radioactive isotope a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

18. A combination for killing or inactivating C. difficile spores comprising:
an aptamer of capable of specifically binding to C. difficile; and
a sporicidal agent agent.

19. The combination according to claim 19, wherein the aptamer comprises:
(a) a nucleic acid sequence selected from any one of the nucleic acid sequences as set forth in any of SEQ ID NOs: 1 to 14, 23 to 39, or 43 to 55;
(b) a nucleic acid sequence having at least 90% sequence identity with any one of the nucleic acid sequences as set forth in any of SEQ ID NOs: 1 to 14, 23 to 39, or 43 to 55;
(c) a nucleic acid sequence having at least about 20 consecutive nucleotides of any one the nucleic acid sequences as set forth in any of SEQ ID NOs: 1 to 14, 23 to 39, or 43 to 55;
(d) a nucleic acid sequence having at least about 20 consecutive nucleotides of a sequence having at least 85% identity with any one of SEQ ID NOs: 1 to 14, 23 to 39, or 43 to 55, or
(e) a combination of any of (a) to (d).

20. The combination according to claim 19, wherein the sporicidal agent comprises peracetic acid and/or an agent capable of generating peracetic acid.
